(19) United States
(12) Patent Application Publication
(10) Pub. No.: US 2022/0310270 A1
(43) Pub. Date: Sep. 29, 2022

(54) INFECTION RISK DETERMINATION SYSTEM, INFECTION RISK DETERMINATION METHOD AND COMPUTER-READABLE MEDIUM

(71) Applicant: Asahi Kasei Microdevices Corporation, Tokyo (JP)

(72) Inventors: Yusaku KARATSU, Tokyo (JP); Takaaki FURUYA, Tokyo (JP)

(21) Appl. No.: 17/702,780

(22) Filed: Mar. 23, 2022

(30) Foreign Application Priority Data
Mar. 26, 2021 (JP) 2021-053635
Mar. 11, 2022 (JP) 2022-037727

(51) Int. Cl. G16H 50/80; G16H 50/30; G10L 25/30

(52) U.S. Cl. CPC G16H 50/80; G16H 50/30; G10L 25/30

(57) ABSTRACT

Provided is an infection risk determination system including: a determination apparatus, including a determination unit configured to determine an infection risk degree that living bodies present in a determination target will be infected with an infection source present in the determination target, based on a carbon dioxide concentration in the determination target with an internal space for accommodating a gas containing carbon dioxide and environmental information in the determination target; and a risk control unit, configured to control at least one of an airstream, a temperature, a humidity, an intensity of ultraviolet radiation, and an amount of substances in a gas in the internal space based on a determination result of the infection risk degree; and a display unit, configured to display a control state by the risk control unit.

The contents of the following Japanese patent application(s) are incorporated herein by reference:
NO. 2021-053635 filed in JP on Mar. 26, 2021
NO. 2022-037727 filed in JP on Mar. 11, 2022

### BACKGROUND

### 1. Technical Field

The present invention relates to an infection risk determination system, an infection risk determination method and a computer-readable medium.

### 2. Related Art

Patent Document 1 discloses “acquires, from each of a carbon dioxide sensor installed in a target facility and a biological sensor installed at a sleeping place in the target facility, a carbon dioxide concentration in the target facility and a biological signal of a target at the sleeping place” (paragraph 0006).

### PRIOR ART DOCUMENT

### Patent Document

[Patent Document 1] Japanese Patent Application Publication No. 2020-071621

The above summary of the invention does not enumerate all of the features of the invention. Subcombinations of these feature groups may also be inventive.

### BRIEF DESCRIPTION OF THE DRAWINGS

FIG. 1 shows an example of a determination target 500 according to one embodiment of the present invention.

FIG. 2 shows an example of a case, when seeing the determination target 500 shown in FIG. 1 in a direction from a ceiling portion 506 toward a floor portion 502.

FIG. 3 is a block diagram showing an example of an infection risk determination system 200 according to one embodiment of the present invention.

FIG. 4 is a block diagram showing another example of the infection risk determination system 200 according to one embodiment of the present invention.

FIG. 5 is a block diagram showing another example of the infection risk determination system 200 according to one embodiment of the present invention.

FIG. 6 is a block diagram showing another example of the infection risk determination system 200 according to one embodiment of the present invention.

FIG. 7 is a block diagram showing another example of the infection risk determination system 200 according to one embodiment of the present invention.

FIG. 8 is a block diagram showing another example of the infection risk determination system 200 according to one embodiment of the present invention.

FIG. 9 shows another example of the case, when seeing the determination target 500 shown in FIG. 1 in the direction from the ceiling portion 506 toward the floor portion 502.

FIG. 10 is a block diagram showing another example of the infection risk determination system 200 according to one embodiment of the present invention.

FIG. 11 shows another example of the determination target 500 according to one embodiment of the present invention.

FIG. 12 is a block diagram showing another example of the infection risk determination system 200 according to one embodiment of the present invention.

FIG. 13 shows another example of the determination target 500 according to one embodiment of the present invention.

FIG. 14 is a block diagram showing another example of the infection risk determination system 200 according to one embodiment of the present invention.

FIG. 15 is a block diagram showing another example of the infection risk determination system 200 according to one embodiment of the present invention.

FIG. 16 is a block diagram showing another example of the infection risk determination system 200 according to one embodiment of the present invention.

FIG. 17 is a block diagram showing another example of the infection risk determination system 200 according to one embodiment of the present invention.

FIG. 18 shows an example of a display aspect on a display unit 30.

FIG. 19 shows an example of a derivation method of a determination result Rd.

FIG. 20 shows an example of a display aspect on a mobile terminal 110.

FIG. 21 shows an example of a determination apparatus 100 according to one embodiment of the present invention.

FIG. 22 is a flowchart showing an example of a determination method according to one embodiment of the present invention.

FIG. 23 is a flowchart showing another example of the determination method according to one embodiment of the present invention.

FIG. 24 is a flowchart showing another example of the determination method according to one embodiment of the present invention.

FIG. 25 is a flowchart showing another example of the determination method according to one embodiment of the present invention.

FIG. 26 shows an example of a computer 2200 in which the determination apparatus 100 according to one embodiment of the present invention may be entirely or partially embodied.

### DESCRIPTION OF EXEMPLARY EMBODIMENTS

Hereinafter, the present invention will be described through embodiments of the invention, but the following embodiments do not limit the invention according to the claims. In addition, not all combinations of features described in the embodiments are essential to the solution of the invention.

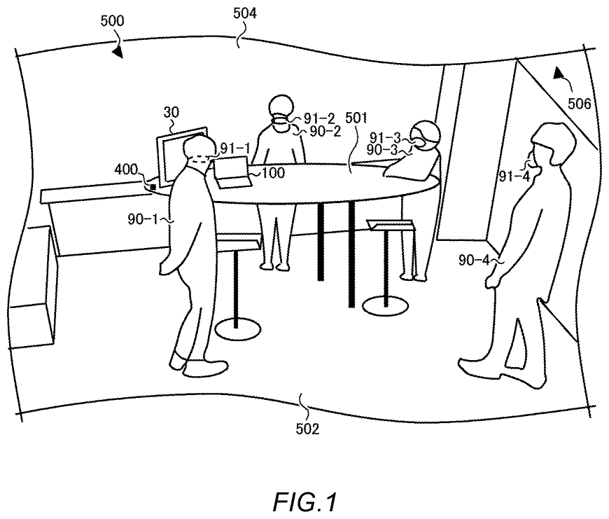

FIG. 1 shows an example of a determination target 500 according to one embodiment of the present invention. In the determination target 500, an infection source (which will be described later) is present. The determination target 500 is a target object that is a target of determination as to an infection risk degree of being infected with the infection source. The target object is an indoor room, for example. In the present example, the determination target 500 is a room having a floor portion 502, a wall portion 504, and a ceiling portion 506.

In the determination target 500, one or a plurality of living bodies 90 are present. In the present example, there are four living bodies 90 (living body 90-1 to living body 90-4) in the determination target 500. The living body 90 is a living organism that repeats exhalation from the lungs and inhalation to the lungs. In the present example, the living body 90 is a human being. In the present example, a nose or mouth of the living body 90 is covered with a mask 91.

In the present example, a determination apparatus 100 and a display unit 30 are arranged in an internal space (which will be described later) surrounded by the floor portion 502, the wall portion 504, and the ceiling portion 506. The determination apparatus 100 is configured to determine a risk degree that the living body 90 will be infected with an infection source (which will be described later) present in the determination target 500. The display unit 30 is, for example, a monitor, a display, or the like. The determination apparatus 100 and the display unit 30 may also be arranged outside the internal space surrounded by the floor portion 502, the wall portion 504, and the ceiling portion 506.

In the determination target 500, one or a plurality of carbon dioxide concentration acquisition units 400 may be arranged. In the present example, the carbon dioxide concentration acquisition unit 400 is arranged on a desk 501. In the present example, the determination apparatus 100 and the display unit 30 are placed on the desk 501. The carbon dioxide concentration acquisition units 400 is, for example, a CO₂ (carbon dioxide) sensor. The carbon dioxide concentration acquisition units 400 may acquire the carbon dioxide concentration from the environmental information Ie (described below). For example, the carbon dioxide concentration acquisition units 400 may also acquire the carbon dioxide concentration from the image captured by the image capturing unit 80 (described below). The carbon dioxide concentration acquired from the image may be an estimate value of the carbon dioxide concentration.

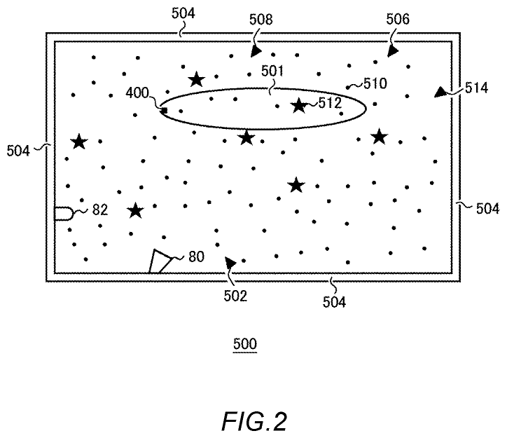

FIG. 2 shows an example of a case, when seeing the determination target 500 shown in FIG. 1 in a direction from the ceiling portion 506 toward the floor portion 502. However, in FIG. 2, the living body 90, the mask 91, the determination apparatus 100, and the display unit 30 shown in FIG. 1 are omitted. In the present example, the determination target 500 has an internal space 508. The internal space 508 is a space partitioned by the wall portion 504 and the like, and is a space isolated from an outside of the internal space 508. In the present example, the internal space 508 is a closed space surrounded by the floor portion 502, the wall portion 504, and the ceiling portion 506.

In the present example, a gas containing CO₂ (carbon dioxide) 510 is accommodated in the internal space 508. The gas may be air. The air may contain CO₂ (carbon dioxide) 510. The carbon dioxide concentration acquisition unit 400 (refer to FIG. 1) is configured to measure a concentration of CO₂ (carbon dioxide) 510 in the internal space 508.

In the determination target 500, an infection source 512 is present. In the present example, the infection source 512 is present in the internal space 508. in FIG. 2, the infection source 512 is denoted with a star mark. The infection source 512 is, for example, a virus, a bacterium, or the like. The infection source 512 may be contained in the gas accommodated in the internal space 508 or may also be attached on the wall portion 504 and the like. The infection source 512 may be a SARS-CoV-2 virus. The SARS-CoV-2 virus is a so-called new corona virus. In a case where the living body 90 (refer to FIG. 1) is infected with the infection source 512, the infection source 512 emitted by the exhalation of the living body 90 may also be present in the determination target 500.

In the determination target 500, an image capturing unit 80 may be arranged. The image capturing unit 80 is, for example, a camera. The image capturing unit 80 may also be a thermography camera configured to measure a body temperature of the living body 90. In the present example, the image capturing unit 80 is provided on the wall portion 504. The image capturing unit 80 is configured to capture an image of the determination target 500. The image capturing unit 80 may be configured to capture an image of the internal space 508. FIG. 1 may be an image captured by the image capturing unit 80. The image capturing unit 80 may be configured to capture a still image or may also be configured to capture a moving image.

In the determination target 500, a voice acquisition unit 82 may be arranged. The voice acquisition unit 82 is, for example, a microphone. In the present example, the voice acquisition unit 82 is provided on the wall portion 504. The voice acquisition unit 82 may also be provided to the display unit 30 (refer to FIG. 1) arranged in the internal space 508. The voice acquisition unit 82 is configured to acquire a sound of the living body 90.

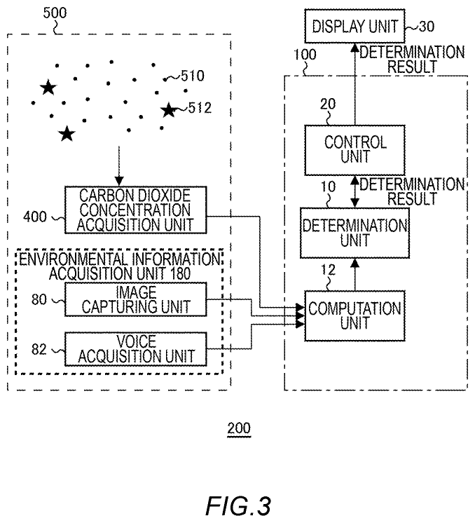

FIG. 3 is a block diagram showing an example of an infection risk determination system 200 according to one embodiment of the present invention. In the present example, the infection risk determination system 200 comprises the determination apparatus 100, the display unit 30, the carbon dioxide concentration acquisition unit 400 and an environmental information acquisition unit 180. In FIG. 3, a range of the determination apparatus 100 is shown by a dashed-dotted line frame.

The determination apparatus 100 comprises a determination unit 10. The determination unit 10 is configured to determine an infection risk degree that the living body 90 (refer to FIG. 1) will be infected with the infection source 512, based on a concentration of CO₂ (carbon dioxide) 510 in the determination target 500 and environmental information Ie (which will be described later) in the determination target 500. The infection risk degree is referred to as infection risk degree Ifr. The infection risk degree Ifr may be a degree of risk that the living body 90 in the determination target 500 will be infected with the infection source 512 through mediation of a gas containing CO₂ (carbon dioxide) 510. The infection risk degree Ifr may also be a degree of risk (cluster level) that a plurality of living bodies 90 in the determination target 500 will be group (cluster)-infected with the infection source 512 by mediation of the gas.

In the present example, the concentration of CO₂ (carbon dioxide) 510 in the determination target 500 is measured by the carbon dioxide concentration acquisition unit 400. In the present example, the environmental information Ie (which will be described later) in the determination target 500 is acquired by the environmental information acquisition unit 180. The concentration information about CO₂ (carbon dioxide) 510 measured by the carbon dioxide concentration acquisition unit 400 and the environmental information Ie (which will be described later) acquired by the environmental information acquisition unit 180 may be wirelessly transmitted to the determination unit 10.

The determination unit 10 may be a CPU (Central Processing Unit). The determination apparatus 100 may be a computer including the CPU, a memory, an interface, and the like. The determination apparatus 100 may also be a portable computer such as a tablet. The determination unit 10 may be configured to output a determination result of the infection risk degree Ifr. The determination result is referred to as determination result Rd.

The determination apparatus 100 may comprise a computation unit 12. The computation unit 12 is configured to compute the concentration of CO₂ (carbon dioxide) 510 in the determination target 500 and the environmental information Ie (which will be described later) in the determination target 500, and to output a computation result to the determination unit 10. Note that, the computation unit 12 may also be included in the determination unit 10. In a case where the computation unit 12 is included in the determination unit 10, the determination unit 10 may be configured to compute the concentration of CO₂ (carbon dioxide) 510 in the determination target 500 and the environmental information Ie (which will be described later) in the determination target 500, and to determine the infection risk degree Ifr, based on a computation result.

The computation unit 12 may be a CPU (Central Processing Unit). In a case where the computation unit 12 is included in the determination unit 10, the determination unit 10 and the computation unit 12 may be one CPU.

The computation unit 12 may be configured to compute the concentration of CO₂ (carbon dioxide) 510 in the determination target 500 and the environmental information Ie in the determination target 500, according to Expression 1, Expression 2 or Expression 3. A computation result may be output to the determination unit 10.

$$[ Expression 1 ]$$

$$P = {\sum }_{i \neq j} {P}_{1}^{i} {P}_{2}^{i j} \qquad ( 1 )$$

$$[ Expression 2 ]$$

$$R = {\sum }_{i \neq j} \frac{{P}_{2}^{i j}}{N} \qquad ( 2 )$$

$$[ Expression 3 ]$$

$$R = {\sum }_{i \neq j} {p}^{i} {P}_{2}^{i j} \qquad ( 3 - 1 )$$

$${\sum }_{i} {p}^{i} = 1 \qquad ( 3 - 2 )$$

P in the Expression 1 is an infection probability of being infected with the infection source 512 in the determination target 500. R in the Expression 2 is a number of infected persons who newly increase when there is one infected person infected with the infection source 512 in the determination target 500. The number of infected persons R is a number of infected persons who are newly reproduced from one infected person in the determination target 500. Since the number of infected persons R increases monotonically with respect to the number of living bodies 90 present in the determination target 500, the number of infected persons may depend on number information In.

P₁ⁱ in the Expression 1 is a probability that the i^{th} living body 90-i is infected with the infection source 512. The probability P₁ⁱ may depend on statistical information Ist (which will be described later) regarding an infection status where the living body 90 is infected with the infection source 512, infection information Ifi (which will be described later) and the environmental information Ie (which will be described later) such as body temperature information It (which will be described later) or sound information Iv (which will be described later).

P₂^{ij} in the Expression 1 to the Expression 3 is a probability that the i^{th} living body 90-i infected with the infection source 512 will infect the j^{th} living body 90-j. pᵢ in the Expression 3 is a probability that, when there is one infected person infected with the infection source 512 in the determination target 500, the i^{th} living body 90-i is infected with the infection source 512. The probability p may depend on the statistical information Ist (which will be described later) regarding an infection status where the living body 90 is infected with the infection source 512, the infection information Ifi (which will be described later) and the environmental information Ie (which will be described later) such as body temperature information It (which will be described later) or sound information Iv (which will be described later).

The computation unit 12 may be configured to compute the probability P₂^{ij} according to Expression 4.

[Expression 4]

p₂^{ij}=1−exp(−n^{ij} (4)

n^{ij} in the Expression 4 is an amount that the j^{th} living body 90-j inhales infectious particles IPa emitted by the i^{th} living body 90-i. The amount may be a trial computation amount.

The computation unit 12 may be configured to compute in the Expression 4, according to Expression 5.

[Expression 5]

n^{ij}=∫ₜ_{ⱼ}c^{ij}B^{j}mᵢₙ^{j}dt (5)

C^{ij} in the Expression 5 is an estimate value of a concentration around a nose or mouth of the j^{th} living body 90-j, and is an estimate value of a concentration of the infectious particles IPa emitted by the i^{th} living body 90-i. B in the Expression 5 is a respiratory volume of the j^{th} living body 90-j. The respiratory volume of the living body 90 may be an amount of a gas that is inhaled or emitted per unit time by the living body 90. As the respiratory volume of the living body 90 increases, the amount of the infectious particles IPa that are inhaled by the living body 90 also increases. Therefore, B^{j} may depend on motion information Im (which will be described later) or exposure information Ip (which will be described later).

mᵢₙ^{j} in the Expression 5 is a correction coefficient for correcting an amount of the infectious particles IPa that are inhaled by the j^{th} living body 90-j. mᵢₙ^{j} may be a correction coefficient due to inhalation inhibition of the infectious particles IPa of the j^{th} living body 90-j by the mask of the j^{th} living body 90-j. Since the amount of the infectious particles IPa that are inhaled by the living body 90 may depend on a type of the mask, mᵢₙ^{j} may depend on the exposure information Ip. tⱼ in the Expression 5 is a variable indicative of a time. tⱼ is a time during which the j^{th} living body 90-j is exposed to the infectious particles IPa in the determination target 500. tⱼ may depend on staying time information Is (which will be described later).

The computation unit 12 may be configured to compute c^{ij} in the Expression 5, according to Expression 6.

$$[ Expression 6 ]$$

$${c}^{i j} = {C}_{L}^{i j} \frac{{E}_{q}^{i}}{{Q}^{j}} {m}_{e x}^{i} \qquad ( 6 )$$

C_{L}^{ij} in the Expression 6 is a correction coefficient depending on a distance between the i^{th} living body 90-i and the j^{th} living body 90-j or respective locations of the living body 90-i and the living body 90-j. A concentration of the infectious particles IPa that are emitted by the living body 90-i is likely to decrease as the distance between the living body 90-i and the living body 90-j increases. For this reason, C_{L}^{ij} may be a function that decreases as the distance increases. For this reason, C_{L}^{ij} may depend on location information IL (which will be described later) or distance information Id (which will be described later).

Eⁱ_{q} in the Expression 6 is an amount of the infectious particles IPa that are emitted per unit time by the i^{th} living body 90-i. The amount of the infectious particles IPa that are emitted per unit time by the living body 90 may depend on an increase in sound-producing amount or an increase in sound-producing time of the living body 90. The amount of the infectious particles IPa that are emitted per unit time by the living body 90 may depend on a quantity of motion of the living body 90. For this reason, Eⁱ_{q} may depend on sound information Iv or motion information Im.

Q^{j} in the Expression 6 is a volume of a gas in the internal space 508 that is emitted per unit time by an emission unit 509 (which will be described later) of the determination target 500, in the determination target 500 where the j^{th} living body 90-j is present. For this reason, Q^{j} may depend on airstream information Iaf. mₑₓⁱ in the Expression 6 is a correction coefficient for correcting an amount of the infectious particles IPa that are emitted by the j^{th} living body 90-j. mₑₓⁱ may be a correction coefficient due to emission inhibition of the infectious particles IPa of the j^{th} living body 90-j by the mask of the j^{th} living body 90-j. Since the amount of the infectious particles IPa that are emitted by the living body 90 may depend on a type of the mask, mₑₓⁱ may depend on the exposure information Ip.

The computation unit 12 may be configured to compute Q^{j} in the Expression 6, according to Expression 7.

$$[ Expression 7 ]$$

$${Q}^{j} = \frac{Δ {C}^{j}}{{NE}_{{CO}_{2}}^{j}} \qquad ( 7 )$$

Δc^{j} in the expression 7 is a difference between a concentration of CO₂ (carbon dioxide) 510 around the j^{th} living body 90-j and a concentration of CO₂ (carbon dioxide) 510 contained in a gas outside the internal space 508. For this reason, ΔC^{j} may depend on a concentration of CO₂ (carbon dioxide) 510 in the internal space 508 or a distribution of the concentration. Note that, the distribution of the concentration may be acquired by a distribution acquisition unit 14, which will be described later.

N in the Expression 7 is a number of the living bodies 90 in the determination target 500. For this reason, N may depend on number information In (which will be described later). E_{CO2}^{j} in the Expression 7 is an emission amount of CO₂ (carbon dioxide) 510 that is emitted per unit time by the j^{th} living body 90-j. The emission amount of CO₂ (carbon dioxide) 510 that is emitted per unit time by the living body 90 depends on a vital capacity of the living body 90. For this reason, in a case where the living body 90 is a human being, E_{CO2}^{j} may depend on at least one of a sex, an age, a height and a weight of the human being. The emission amount of CO₂ (carbon dioxide) 510 that is emitted per unit time by the living body 90 may depend on a quantity of motion of the living body 90. For this reason, E_{CO2}^{j} may depend on motion information Im (which will be described later).

The determination apparatus 100 may comprise a control unit 20. The control unit 20 may be a CPU (Central Processing Unit). The determination unit 10, the computation unit 12 and the control unit 20 may also be one CPU. The control unit 20 may be configured to transmit a control signal for controlling display of the display unit 30 to the display unit 30.

The environmental information acquisition unit 180 is configured to acquire environmental information in the determination target 500. The environmental information is referred to as environmental information Ie. The environmental information Ie is information regarding the living body 90, and is information capable of affecting determination as to the infection risk degree of being infected with the infection source 512 in the determination target 500. The environmental information Ie may include at least one of sound information about the living body 90, number information about the living body 90, body temperature information about the living body 90, exposure information about a nose or mouth of the living body 90, distance information among a plurality of living bodies 90, location information about the living body 90, staying time information about the living body 90, and motion information about the living body 90.

Since the living body 90 emits CO₂ (carbon dioxide) by exhalation, the concentration of CO₂ (carbon dioxide) 510 in the determination target 500 is more likely to increase as the internal space 508 (refer to FIG. 2) is smaller. The concentration of CO₂ (carbon dioxide) 510 in the determination target 500 is more likely to increase as an isolation degree that the internal space 508 is isolated from an outside of the internal space 508 is higher. The infection risk degree Ifr is likely to depend on the concentration of CO₂ (carbon dioxide) 510 in the determination target 500. For this reason, the smaller the internal space 508 is and the higher the above-described isolation degree is, the infection risk degree Ifr is more likely to increase. Note that, the high isolation degree indicates that a ventilation state in the internal space 508 is poor.

The sound information about the living body 90 is referred to as sound information Iv. The sound information Iv indicates information about a sound that is produced from the living body 90. The sound that is produced from the living body may refer to a sound that is produced from a sound-producing organ (mainly, a mouth and a throat). Information about the sound that is produced from the living body 90 may include at least one of a voice sound, a coughing sound, and a sneezing sound, which are produced from the living body 90. The sound information Iv may refer to at least one of loudness and frequency of a sound that is produced from the living body 90 or may also refer to a voiceprint of the living body 90. The sound information Iv may include sex information about sound of the living body 90.

The determination unit 10 may be configured to determine the infection risk degree Ifr, based on the concentration of CO₂ (carbon dioxide) 510 in the determination target 500 and the sound information Iv. In a case where the living body 90 (refer to FIG. 1) is infected with the infection source 512, the infection source 512 emitted by exhalation of the living body 90 may be infectious particles. The infectious particles are referred to as infectious particles IPa. The infectious particles IPa are likely to be emitted by sound production of the living body 90. When the loudness of sound of the living body 90 increases, an increase rate of the infectious particles IPa is likely to be larger than an increase rate of the loudness of sound of the living body 90. For this reason, the determination unit 10 can determine the infection risk degree Ifr, based on the concentration of CO₂ (carbon dioxide) 510 and the sound information Iv.

The number information about the living body 90 is referred to as number information In. The number information In refers to a number of the living bodies 90 present in the determination target 500.

The determination unit 10 may be configured to determine the infection risk degree Ifr, based on the concentration of CO₂ (carbon dioxide) 510 in the determination target 500 and the number information In. Since the living body 90 emits CO₂ (carbon dioxide) by exhalation, the concentration of CO₂ (carbon dioxide) 510 in the determination target 500 is more likely to increase as the number of the living bodies 90 present in the determination target 500 is larger. For this reason, the determination unit 10 can determine the infection risk degree Ifr, based on the concentration of CO₂ (carbon dioxide) 510 and the number information In.

The body temperature information about the living body 90 is referred to as body temperature information It. The body temperature information It may be information about a body surface temperature of the living body 90 present in the determination target 500 or may also be information about an internal body temperature. In a case where the body temperature information It is the information about the body surface temperature, the body temperature information It may also be distribution information about body surface temperatures, in which a distribution of the body surface temperatures is displayed in a two-dimensional shape.

The determination unit 10 may be configured to determine the infection risk degree Ifr, based on the concentration of CO₂ (carbon dioxide) 510 in the determination target 500 and the body temperature information It. In a case where the living body 90 (refer to FIG. 1) is infected with the infection source 512, the body temperature of the living body 90 is likely to rise. For this reason, the determination unit 10 can determine the infection risk degree Ifr, based on the concentration of CO₂ (carbon dioxide) 510 and the body temperature information It.

The exposure information about a nose or mouth of the living body 90 is referred to as exposure information Ip. The exposure information Ip may be information as to whether at least one of a nose and a mouth of the living body 90 is exposed. The exposure information Ip may also be information about airstream based on exhalation from the nose and the mouth of the living body 90 in a case where the nose and the mouth of the living body 90 are not exposed.

The determination unit 10 may be configured to determine the infection risk degree Ifr, based on the concentration of CO₂ (carbon dioxide) 510 in the determination target 500 and the exposure information Ip. In a case where at least one of the nose and the mouth of the living body 90 is exposed, when the living body 90 is infected with the infection source 512, the living body 90 is likely to emit the infectious particles IPa by voice production and to inhale the infectious particles IPa by inhalation. For this reason, the determination unit 10 can determine the infection risk degree Ifr, based on the concentration of CO₂ (carbon dioxide) 510 and the exposure information Ip.

The distance information among the plurality of living bodies 90 is referred to as distance information Id. The distance information Id may refer to a distance between a nose or mouth of one living body 90 (for example, the living body 90-3 in FIG. 1) and a nose or mouth of another living body 90 (for example, the living body 90-4 in FIG. 1). In a case where three or more living bodies 90 are present in the determination target 500, the distance information Id may include a plurality of distances Id between a plurality of types of two living bodies 90 selected from the three or more living bodies 90.

The determination unit 10 may be configured to determine the infection risk degree Ifr, based on the concentration of CO₂ (carbon dioxide) 510 in the determination target 500 and the distance information Id. In a case where one living body 90 is infected with the infection source 512, the closer the distance between one living body 90 and another living body 90 is, the easier another living body 90 can inhale the infectious particles IPa, which are emitted by one living body 90, by inhalation. For this reason, the determination unit 10 can determine the infection risk degree Ifr, based on the concentration of CO₂ (carbon dioxide) 510 and the distance information Id.

The location information about the living body 90 is referred to as location information IL. The location information IL refers to location information about the living body 90 in the determination target 500. The location information about the living body 90 in the determination target 500 refers to location information about the living body 90 in the internal space 508 (refer to FIG. 2). The location information IL may refer to location information about a nose or mouth of the living body 90.

The determination unit 10 may be configured to determine the infection risk degree Ifr, based on the concentration of CO₂ (carbon dioxide) 510 in the determination target 500 and the location information IL. The air in the internal space 508 (refer to FIG. 2) is likely to stay near the wall portion 504. For this reason, when the location information about the living body 90 infected with the infection source 512 is near the wall portion 504, the infectious particles IPa emitted by the living body 90 are likely to stay near the wall portion 504. For this reason, the determination unit 10 can determine the infection risk degree Ifr, based on the concentration of CO₂ (carbon dioxide) 510 and the location information IL.

The staying time information about the living body 90 is referred to as staying time information Is. The staying time information Is refers to staying time information about the living body 90 in the determination target 500. The staying time information Is may be an elapsed time from a time point when the living body 90 enters the internal space 508 from an outside of the internal space 508, or may also be a time from a time point when the living body enters the internal space 508 to a time point when the living body goes to an outside of the internal space 508.

The determination unit 10 may be configured to determine the infection risk degree Ifr, based on the concentration of CO₂ (carbon dioxide) 510 in the determination target 500 and the staying time information Is. In a case where the living body 90 infected with the infection source 512 stays in the determination target 500, an amount of the infectious particles IPa emitted by the living body 90 is likely to depend on a time during which the living body 90 stays in the determination target 500. For this reason, the determination unit 10 can determine the infection risk degree Ifr, based on the concentration of CO₂ (carbon dioxide) 510 and the staying time information Is.

The motion information about the living body 90 is referred to as motion information Im. The motion information Im refers to motion information about the living body 90 in the determination target 500. The motion information Im may be information about metabolic equivalents (METs) or movement of the living body 90. The metabolic equivalents (METs) are an amount obtained by standardizing an amount of O₂ (oxygen) that is consumed by the living body 90 when the living body 90 is in a motion state with an amount of O₂ (oxygen) that is consumed by the living body 90 when the living body 90 is in a resting state. The movement information about the living body 90 may information about movement of a body of the living body 90.

The determination unit 10 may be configured to determine the infection risk degree Ifr, based on the concentration of CO₂ (carbon dioxide) 510 in the determination target 500 and the motion information Im. When a quantity of motion of the living body 90 increases, a cycle of exhalation of the living body 90 is likely to be short and a total amount of exhalation of the living body 90 during a predetermined time is likely to increase. The motion information Im may also be information about a cycle of exhalation of the living body 90. When a total amount of exhalation or a cycle of exhalation of the living body 90 increases, an emission amount of the infectious particles IPa is likely to increase. When the quantity of motion of the living body 90 increases, an increase rate of the infectious particles IPa is likely to be larger than an increase rate of the quantity of motion of the living body 90. For this reason, the determination unit 10 can determine the infection risk degree Ifr, based on the concentration of CO₂ (carbon dioxide) 510 and the motion information Im.

In a case where the living body 90 is infected with the infection source 512, the amount of infectious particles IPa that are emitted by the living body 90 is likely to increase as the sound of the living body 90 increases. When the quantity of motion of the living body 90 increases and the sound is being produced from the living body 90, the emission amount of the infectious particle IPa is more likely to increase. For this reason, the determination unit 10 can determine the infection risk degree Ifr, based on the concentration of CO₂ (carbon dioxide) 510, the motion information Im and the sound information Iv.

The determination unit 10 may also be configured to determine the infection risk degree Ifr, based on the plurality of information selected from the sound information Iv, the number information In, the body temperature information It, the exposure information Ip, the distance information Id, the location information IL, the staying time information Is and the motion information Im, and the concentration of CO₂ (carbon dioxide) 510 in the determination target 500. The infection risk degree Ifr is determined based on the plurality of information and the concentration of CO₂ (carbon dioxide) 510, so that the infection risk degree Ifr is likely to be determined more correctly, as compared to a case where the infection risk degree Ifr is determined based on the one information and the concentration of CO₂ (carbon dioxide) 510.

In the present example, the environmental information acquisition unit 180 includes an image capturing unit 80 and a voice acquisition unit 82. The number information In, the body temperature information It, the exposure information Ip, the distance information Id, the location information IL, the staying time information Is and the motion information Im may be information based on an image captured by the image capturing unit 80. The computation unit 12 may be configured to compute at least one of the number information In, the body temperature information It, the exposure information Ip, the distance information Id, the location information IL, the staying time information Is and the motion information Im, based on the image captured by the image capturing unit 80. The determination unit 10 may be configured to determine at least one of the number information In, the body temperature information It, the exposure information Ip, the distance information Id, the location information IL, the staying time information Is and the motion information Im, based on a computation result made by the computation unit 12.

The sound information Iv may be information based on a sound of the living body 90 acquired by the voice acquisition unit 82. The information based on a sound of the living body 90 may refer to information about sound that is produced from the living body 90. As described above, the information about the sound that is produced from the living body 90 may include at least one of a voice sound, a coughing sound, and a sneezing sound, which are produced from the living body 90. The computation unit 12 may be configured to compute the sound information Iv, based on the sound of the living body 90 acquired by the voice acquisition unit 82. The determination unit 10 may be configured to determine the sound information Iv, based on a computation result made by the computation unit 12.

The display unit 30 may be configured to display the determination result Rd. The control unit 20 may be configured to control the display unit 30 to display the determination result Rd on the display unit 30. The determination result Rd may also be displayed on the display unit 30 with information having readability or may be displayed with information having visual recognition. The information having readability is, for example, letters and numbers. The information having visual recognition is, for example, a graph, a figure, and the like. The determination result Rd is displayed on the display unit 30, so that the determination result Rd is recognized by a user of the infection risk determination system 200. The determination result Rd may also be recognized by the user of the infection risk determination system 200, depending on whether the determination apparatus 100 outputs a voice, the determination apparatus 100 vibrates or the determination apparatus emits an odor.

The determination unit 10 may be configured to determine the living body 90, which is an information source of the sound information Iv, among the plurality of living bodies 90, based on the sound of the living body 90 (refer to FIG. 1) acquired by the voice acquisition unit 82. In the example shown in FIG. 1, when the living body 90-2 is producing a voice, the voice acquisition unit 82 can acquire that the sound of the living body 90-2 is a sound from a direction of the living body 90-2. For this reason, the determination unit 10 can determine that the living body 90, which is an information source of the sound information Iv, is the living body 90-2.

The determination unit 10 may also be configured to determine the living body 90, which is an information source of the sound information Iv, among the plurality of living bodies 90, based on an image of the determination target 500 captured by the image capturing unit 80 and a sound of the living body 90 (refer to FIG. 1) acquired by the voice acquisition unit 82. In the example shown in FIG. 1, when the living body 90-2 is producing a voice, the voice acquisition unit 82 can acquire that the sound of the living body 90-2 is a sound from a direction of the living body 90-2.

In a case where the voice acquisition unit 82 acquires a sound of the living body 90-2 as a sound from the direction of the living body 90-2, when the image capturing unit 80 image-captures movement of a mouth of the living body 90-2 and does not image-capture movement of a mouth of the living body 90-3, for example, the determination unit 10 can determine that the living body 90, which is an information source of the sound information Iv, is the living body 90-2. For this reason, the determination unit 10 can determine the living body 90 more correctly, which is an information source of the sound information Iv, as compared to a case where the determination unit 10 determines the living body 90, which is an information source of the sound information Iv, based on a sound of the living body 90 (refer to FIG. 1) without relying on an image of the determination target 500.

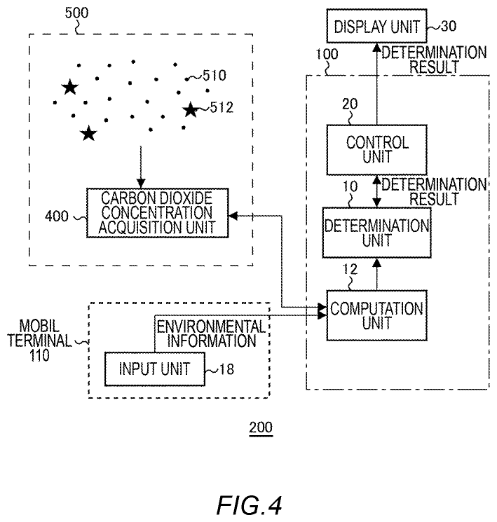

FIG. 4 is a block diagram showing another example of the infection risk determination system 200 according to one embodiment of the present invention. The infection risk determination system 200 of the present example is different from the infection risk determination system 200 shown in FIG. 3, in that the infection risk determination system comprises an input unit 18 without the environmental information acquisition unit 180. The input unit 18 may be provided to a mobile terminal 110 separate from the determination apparatus 100. The mobile terminal 110 may be arranged outside the internal space 508. The mobile terminal 110 may be wirelessly connected to the determination apparatus 100.

The environmental information Ie may also be input by the input unit 18. The user of the infection risk determination system 200 may input the environmental information Ie from the input unit 18. The environmental information Ie may be determined more correctly by the user of the infection risk determination system 200, as compared to being determined based on an image acquired by the image capturing unit 80 or a voice acquired by the voice acquisition unit 82. For example, since the number information In is easily visually recognized by the user of the infection risk determination system 200, the user may input the number information In from the input unit 18.

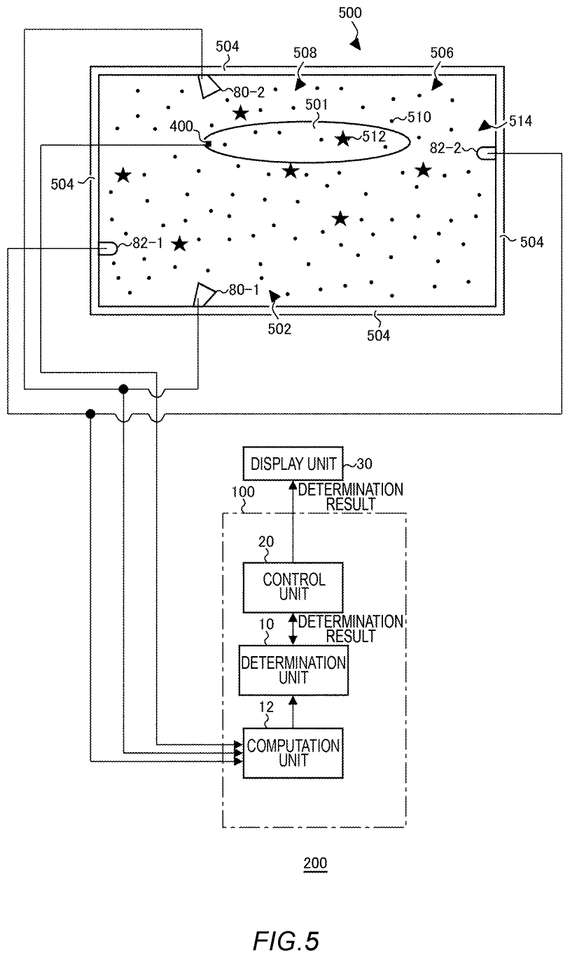

FIG. 5 is a block diagram showing another example of the infection risk determination system 200 according to one embodiment of the present invention. The infection risk determination system 200 may comprise a plurality of image capturing units 80 and a plurality of voice acquisition units 82. In the determination target 500, the plurality of image capturing units 80 and the plurality of voice acquisition units 82 may be arranged. The infection risk determination system 200 of the present example comprises two image capturing units 80 (an image capturing unit 80-1 and an image capturing unit 80-2) and two voice acquisition units 82 (a voice acquisition unit 82-1 and a voice acquisition unit 82-2). In the present example, the two image capturing units 80 and the two voice acquisition units 82 are arranged in the determination target 500.

The determination unit 10 may be configured to determine the living body 90, which is an information source of the sound information Iv, among the plurality of living bodies 90, based on a plurality of images of the determination target 500 captured by the plurality of image capturing units 80 and sounds of the plurality of living bodies 90 (refer to FIG. 1) acquired by the plurality of voice acquisition units 82. The living body 90, which is an information source of the sound information Iv, is determined, based on the plurality of images and the sounds of the plurality of living bodies 90, so that the living body 90, which is an information source of the sound information Iv, is likely to be determined more correctly, as compared to being determined based on one image and a sound of one living body 90. For this reason, the infection risk determination system 200 preferably comprises the plurality of image capturing units 80 and the plurality of voice acquisition units 82.

The environmental information Ie may further include infection information as to whether the living body 90 is infected with the infection source 512. The infection information is referred to as infection information Ifi. In a case where the environmental information Ie includes the infection information Ifi indicating that the living body 90, which is an information source of the sound information Iv, is infected with the infection source 512, the determination unit 10 may be configured to determine the infection risk degree Ifr, based on at least one of a sound-producing time and a sound-producing amount of the living body 90, which is an information source of the sound information Iv.

The sound-producing time of the living body 90 is a time during which the living body 90 emits a sound. The sound-producing time of the living body 90 may be a voice-producing time of the living body 90, or may also be a time during which the living body 90 emits a coughing sound or a sneezing sound. The sound-producing amount of the living body 90 is loudness of the sound that is emitted by the living body 90. The sound-producing amount of the living body 90 may be a voice-producing amount of the living body 90, or may also be a sound volume of coughing or a sound volume of sneezing emitted by the living body 90. The sound-producing amount of the living body 90 may be an amplitude of a sound wave that is emitted by the living body 90, or may also be a maximum value of the amplitude.

In a case where the living body 90, which is an information source of the sound information Iv, is infected with the infection source 512, the living body 90 is more likely to emit the infectious particle IPa by voice production, as compared to a case where the living body 90 is not infected with the infection source 512. In the case where the living body 90 is infected with the infection source 512, the infection risk degree Ifr is likely to depend on at least one of the sound-producing time and the sound-producing amount of the living body 90. For this reason, in a case where the environmental information Ie includes the infection information Ifi, the determination unit 10 can determine the infection risk degree Ifr, based on at least one of a sound-producing time and a sound-producing amount of the living body 90, which is an information source of the sound information Iv.

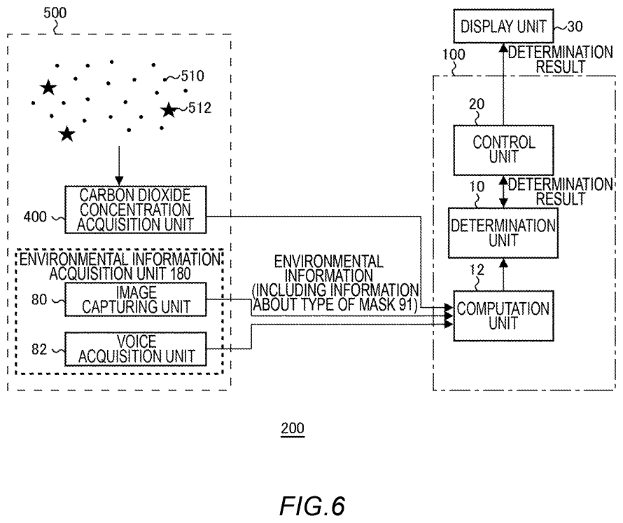

FIG. 6 is a block diagram showing another example of the infection risk determination system 200 according to one embodiment of the present invention. In the present example, the environmental information Ie includes the exposure information Ip, and the exposure information Ip includes information about a type of the mask 91 (refer to FIG. 1) configured to cover a nose or mouth of the living body 90. The type of the mask 91 is, for example, a material, a size, and the like of the mask 91. The determination unit 10 may be configured to correct the determination result Rd of the infection risk degree Ifr, based on the information about the type of the mask 91.

In a case where the nose or mouth of the living body 90 is covered with the mask 91, an emission amount of the infectious particles IPa emitted from the mouth of the living body 90 and an amount of inhalation of the infectious particles IPa inhaled through the mouth or nose of the living body 90 are likely to depend on the type of the mask 91. For example, the infectious particle IPa are more likely to pass through the polyurethane mask 91 than the nonwoven mask 91. For example, the infectious particles IPa are more likely to be emitted into the internal space 508 (refer to FIG. 2) when the mask 91 covers a part of the cheek than when the mask 91 covers the entire cheek of the living body 90. For this reason, the determination unit 10 corrects the determination result Rd of the infection risk degree Ifr, based on the information about the type of the mask 91, so that the infection risk degree Ifr is likely to be determined more correctly.

The information about the type of the mask 91 (refer to FIG. 1) that covers the nose or mouth of the living body 90 may be information based on an image of the determination target 500 captured by the image capturing unit 80. The determination unit 10 may be configured to determine the information about the type of mask 91 that covers the nose or mouth of the living body 90, based on the image captured by the image capturing unit 80.

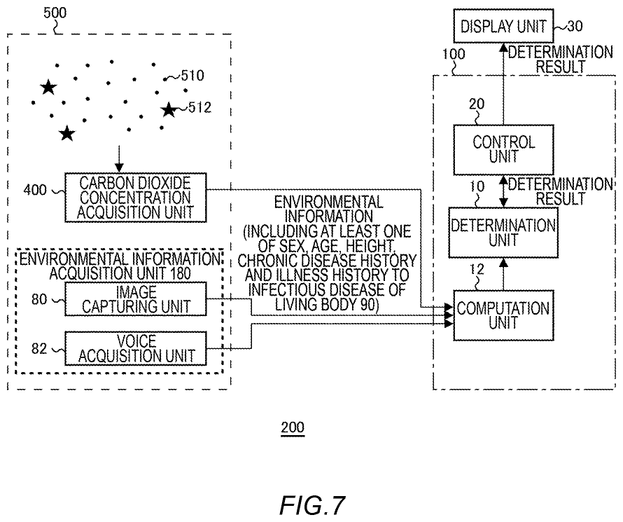

FIG. 7 is a block diagram showing another example of the infection risk determination system 200 according to one embodiment of the present invention. In the present example, the living body 90 is a human being. In the present example, the environmental information Ie further includes at least one of a sex, an age, a height, a chronic disease history, and an illness history to an infectious disease of the living body 90. The determination unit 10 may be configured to correct the determination result Rd of the infection risk degree Ifr, based on at least one of the sex, age, height, chronic disease history and illness history to an infectious disease of the living body 90 (a human being, in the present example).

In a case where the living body 90 is a human being, the infection risk degree Ifr of the living body 90 is likely to depend on the sex, age, height, chronic disease history and illness history to an infectious disease of the living body 90. For example, the infection risk degree Ifr of the seventy-something living body 90 is likely to be higher than the infection risk degree Ifr of the thirty-something living body 90. For example, the infection risk degree Ifr of the living body 90 with a chronic disease history is likely to be higher than the infection risk degree Ifr of the living body 90 without a chronic disease history. For this reason, the determination unit 10 corrects the determination result Rd of the infection risk degree Ifr, based on at least one of the sex, age, height, chronic disease history, and illness history to an infectious disease of the living body 90, so that the infection risk degree Ifr is likely to be determined more correctly.

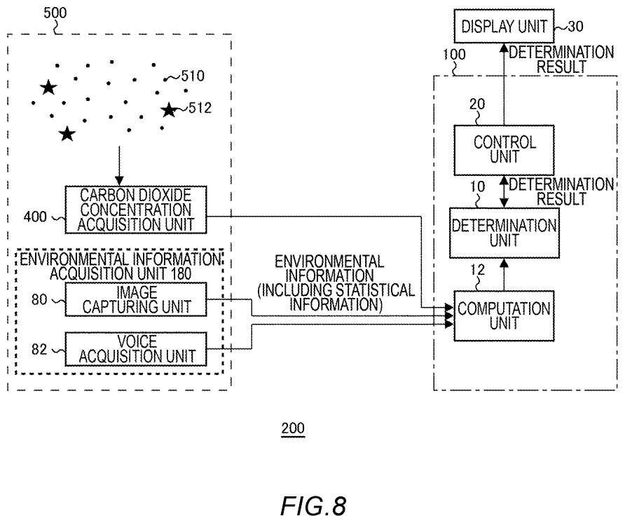

FIG. 8 is a block diagram showing another example of the infection risk determination system 200 according to one embodiment of the present invention. In the present example, the environmental information Ie further includes statistical information regarding an infection status of the living body 90 infected with the infection source 512. The statistical information is referred to as statistical information Ist. The determination unit 10 may be configured to correct the determination result Rd of the infection risk degree Ifr, based on the statistical information Ist.

The statistical information Ist regarding an infection status of the living body 90 infected with the infection source 512 is, for example, a change of the latest infection status to the infection source 512, a current infection status to the infection source 512 in an area of the determination target 500, and the like. In a case where the latest infection status to the infection source 512 is changing to a high-level infestation state, there is a higher probability that the living body 90 becomes infected with the infection source 512 present in the determination target 500, as compared to a case where the infection status is changing to a low-level infestation state. For this reason, the determination unit 10 corrects the determination result Rd of the infection risk degree Ifr, based on the statistical information Ist, so that the infection risk degree Ifr is likely to be determined more correctly.

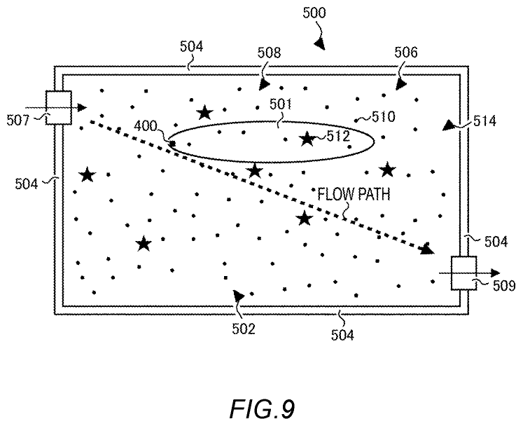

FIG. 9 shows another example of the case, when seeing the determination target 500 shown in FIG. 1 in the direction from the ceiling portion 506 toward the floor portion 502. In the present example, a supply unit 507 and an emission unit 509 are provided to the determination target 500.

The supply unit 507 is configured to supply a gas outside the internal space 508 into the internal space 508. The supply unit 507 is, for example, an air conditioning facility, an air purifier, an air conditioner, or the like. The emission unit 509 is configured to emit a gas in the internal space 508 to an outside of the internal space 508. The emission unit 509 is, for example, a ventilation fan, a ventilation port, or the like.

In a case where the gas outside the internal space 508 is supplied into the internal space 508 by the supply unit 507 and the gas in the internal space 508 is emitted to the outside by the emission unit 509, the gas in the internal space 508 is likely to move in a direction from the supply unit 507 toward the emission unit 509. A flow channel in the direction of the gas is referred to as flow channel Ch.

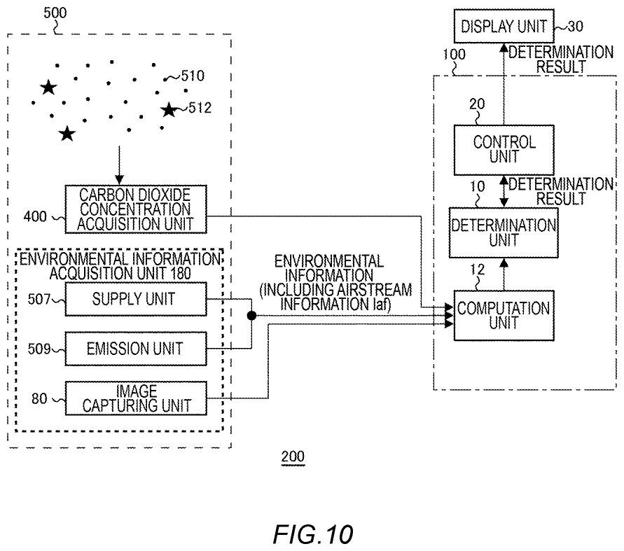

FIG. 10 is a block diagram showing another example of the infection risk determination system 200 according to one embodiment of the present invention. FIG. 10 is an example of a block diagram in a case where the determination target 500 is an example shown in FIG. 9. In the present example, the environmental information Ie further includes airstream information. The airstream information is referred to as airstream information Iaf.

The airstream information Iaf is information about a device that affects airstream in the internal space. The airstream information Iaf may be at least one of information about the supply unit 507 (refer to FIG. 9) and information about the emission unit 509 (refer to FIG. 9). The information about the supply unit 507 may refer to a volume or mass of the gas outside the internal space 508, which is supplied per unit time by the supply unit 507. The information about the supply unit 507 may also refer to location information about the supply unit 507 in the determination target 500. The information about the emission unit 509 may refer to a volume or mass of the gas in the internal space 508, which is emitted per unit time by the emission unit 509. The information about the emission unit 509 may also refer to location information about the emission unit 509 in the determination target 500.

The determination unit 10 may be configured to correct the determination result Rd of the infection risk degree Ifr, based on at least one of the airstream information Iaf and the location information IL about the living body 90. In a case where the gas in the internal space 508 is emitted by the emission unit 509 and the gas outside the internal space 508 is supplied by the supply unit 507, the concentration of CO₂ (carbon dioxide) 510 in the determination target 500 is likely to decrease, and the infectious particles IPa are likely to be emitted to the outside of the internal space 508. For this reason, the infection risk degree Ifr is more likely to decrease, as compared to a case where the gas in the internal space 508 is not emitted and the gas outside the internal space 508 is not supplied. For this reason, the determination unit 10 corrects the determination result Rd of the infection risk degree Ifr, based on the airstream information Iaf, so that the infection risk degree Ifr is likely to be determined more correctly.

In a case where the infectious particles IPa are present in the internal space 508, a distribution of the infectious particles IPa in the internal space 508 is likely to depend on the location of the living body 90 in the internal space 508. For this reason, the determination unit 10 corrects the determination result Rd of the infection risk degree Ifr, based on the location information IL, so that the infection risk degree Ifr is likely to be determined more correctly.

In a case where the gas in the internal space 508 is emitted by the emission unit 509, the gas outside the internal space 508 is supplied by the supply unit 507 and the location information IL about the living body 90 is near the flow channel Ch (refer to FIG. 9), the infection risk degree Ifr that the living body 90 will be infected with the infection source 512 is more likely to decrease, as compared to a case where the location information IL about the living body 90 is distant from the flow channel Ch. For this reason, the determination unit 10 corrects the determination result Rd of the infection risk degree Ifr, based on the airstream information Iaf and the location information IL, so that the infection risk degree Ifr is likely to be determined more correctly. Note that, the location information IL about the living body 90 may be information based on an image captured by the image capturing unit 80.

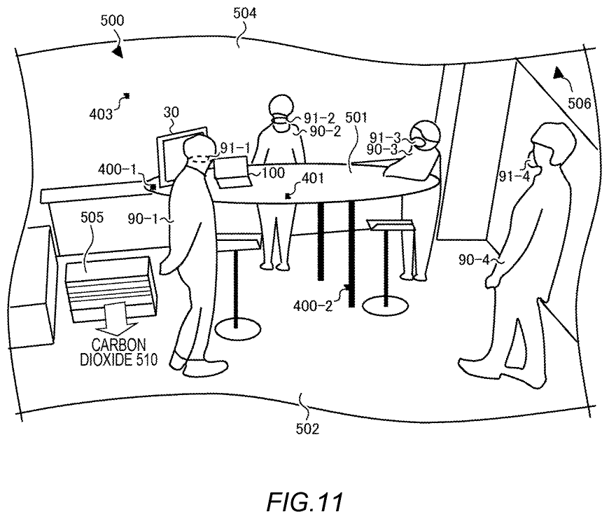

FIG. 11 shows another example of the determination target 500 according to one embodiment of the present invention. In the present example, a CO₂ (carbon dioxide) emission unit 505 configured to emit CO₂ (carbon dioxide) 510 exceeding a predetermined amount in the internal space 508 is arranged in the determination target 500. In this respect, the determination target 500 of the present example is different from the determination target 500 shown in FIG. 2. The CO₂ (carbon dioxide) emission unit 505 is, for example, a stove.

In the determination target 500, a plurality of carbon dioxide concentration acquisition units 400 may be arranged. In the present example, the two carbon dioxide concentration acquisition units 400 are arranged in the determination target 500. Also in this respect, the determination target 500 of the present example is different from the determination target 500 shown in the FIG. 2.

In the present example, the carbon dioxide concentration acquisition unit 400-1 is provided on the desk 501, and the carbon dioxide concentration acquisition unit 400-2 is provided on a leg of the desk 501. A height of the carbon dioxide concentration acquisition unit 400-1 from the floor portion 502 and a height of the carbon dioxide concentration acquisition unit 400-2 from the floor portion 502 may be different.

In the determination target 500, a temperature/humidity sensor 401 and an ultraviolet sensor 403 may be further arranged. Also in this respect, the determination target 500 of the present example is different from the determination target 500 shown in the FIG. 2. The temperature/humidity sensor 401 is configured to measure a temperature and a humidity in the internal space 508. The ultraviolet sensor 403 is configured to measure ultraviolet in the internal space 508. The ultraviolet sensor 403 may be configured to measure at least one of a UVB wave and a UVC wave. In the present example, the temperature/humidity sensor 401 is provided on the desk 501, and the ultraviolet sensor 403 is provided on the wall portion 504.

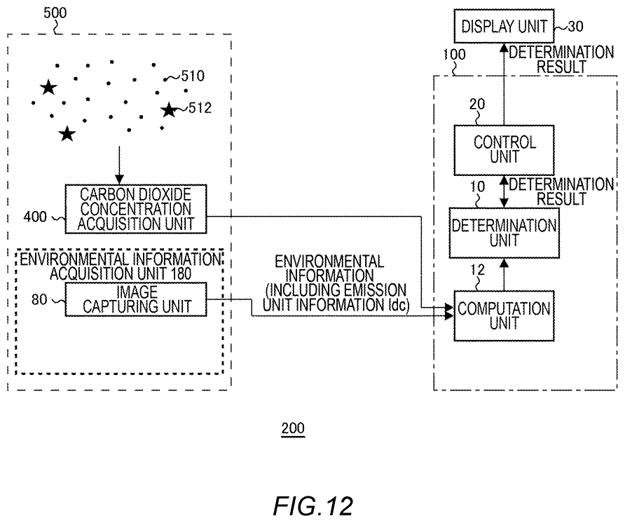

FIG. 12 is a block diagram showing another example of the infection risk determination system 200 according to one embodiment of the present invention. FIG. 12 is an example of a block diagram in a case where the determination target 500 is an example shown in FIG. 11. In the present example, the environmental information Ie further includes information about the carbon dioxide emission unit 505. The information is referred to as emission unit information Idc.

The emission unit information Idc may be information as to whether the CO₂ (carbon dioxide) emission unit 505 is operating. The emission unit information Idc may be information based on an image of the determination target 500 captured by the image capturing unit 80. The determination unit 10 may be configured to determine the emission unit information Idc, based on the image captured by the image capturing unit 80. The determination unit 10 may also be configured to determine the emission unit information Idc, via a wired or wireless communication network.

The determination unit 10 may be configured to correct the determination result Rd of the infection risk degree Ifr, based on the emission unit information Idc. In a case where the emission unit information Idc is information indicating that the CO₂ (carbon dioxide) emission unit 505 is operating, the determination unit 10 may be configured to correct the determination result Rd to a determination result Rd indicating that the infection risk degree Ifr is higher, as compared to a case of information indicating that the emission unit is not operating.

When the CO₂ (carbon dioxide) emission unit 505 is operating, the concentration of CO₂ (carbon dioxide) 510 is likely to be equal to or larger than a predetermined concentration. The predetermined concentration may be a concentration at which a risk that the living body 90 in the determination target 500 will be infected with the infection source 512 can be suppressed. The predetermined concentration is, for example, 1000 ppm.

The determination unit 10 may be configured to correct the determination result Rd of the infection risk degree Ifr, based on a temporal change of the concentration of CO₂ (carbon dioxide) 510. The determination unit 10 may be configured to correct the determination result Rd of the infection risk degree Ifr, when a temporal change rate of the concentration of CO₂ (carbon dioxide) 510 exceeds a predetermined threshold change rate. The case where the predetermined threshold change rate is exceeded is, for example, a case where the carbon dioxide emission unit 505 is operating. When the temporal change rate of the concentration of CO₂ (carbon dioxide) 510 exceeds the predetermined threshold change rate, the infection risk degree Ifr is likely to increase above the concentration of CO₂ (carbon dioxide) 510. For this reason, when the temporal change rate of the concentration of CO₂ (carbon dioxide) 510 exceeds the predetermined threshold change rate, the determination unit 10 corrects the determination result Rd of the infection risk degree Ifr, based on the temporal change rate of the concentration of CO₂ (carbon dioxide) 510, so that the infection risk degree Ifr is likely to be determined more correctly.

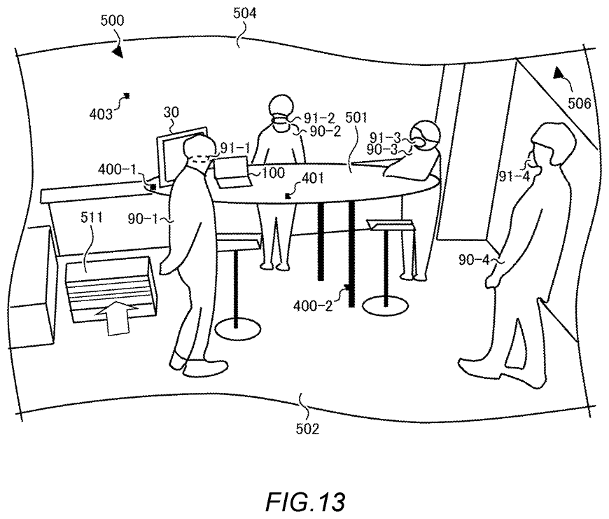

FIG. 13 shows another example of the determination target 500 according to one embodiment of the present invention. In the present example, in the determination target 500, a substance trapping unit 511 is arranged, instead of the CO₂ (carbon dioxide) emission unit 505. In this respect, the infection risk determination system 200 of the present example is different from the infection risk determination system 200 shown the FIG. 12. The substance trapping unit 511 is configured to trap a substance contained in a gas. The gas includes CO₂ (carbon dioxide) 510, and is also accommodated in the internal space 508. The substance trapping unit 511 may trap the infection source 512. The substance trapped by the substance trapping unit 511 may refer to the infectious or infection-promoting substance that controls the increase or decrease in the infection risk degree Ifr that is transmitted through the air of the determination target 500 by the infection source 512 contained in the exhalation of a living body 90 infected with an infectious disease. The substance trapping unit 511 may be configured to trap a substance (mote, dust, virus and the like) contained in the gas by sucking the gas accommodated in the internal space 508. In this respect, the determination target 500 of the present example is different from the determination target 500 shown in FIG. 2. The substance trapping unit 511 is, for example, an air purifier, an air conditioner, or the like.

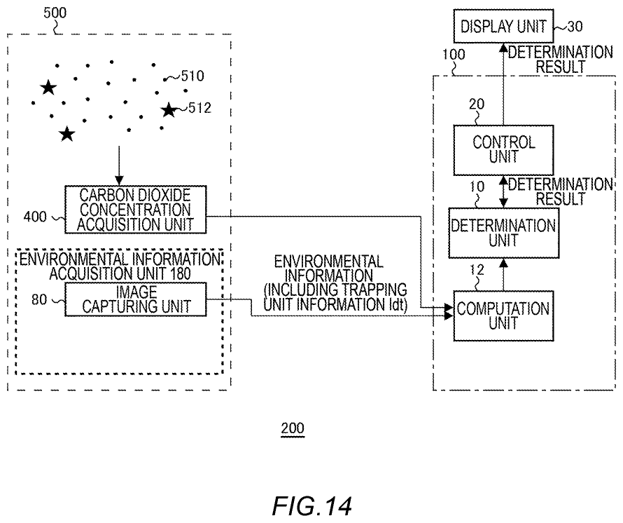

FIG. 14 is a block diagram showing another example of the infection risk determination system 200 according to one embodiment of the present invention. In the present example, the environmental information Ie further includes information about the substance trapping unit 511. In this respect, the infection risk determination system 200 of the present example is different from the infection risk determination system 200 shown the FIG. 12. The information about the substance trapping unit 511 is referred to as trapping unit information Idt.

The trapping unit information Idt may be information as to whether the substance trapping unit 511 is operating. The trapping unit information Idt may be information based on an image of the determination target 500 captured by the image capturing unit 80. The determination unit 10 may be configured to determine the trapping unit information Idt, based on the image captured by the image capturing unit 80. The determination unit 10 may be configured to determine the trapping unit information Idt, via a wired or wireless communication network. The trapping unit information Idt may include information that varies the infection risk degree Ifr.

The determination unit 10 may be configured to correct the determination result Rd of the infection risk degree Ifr, based on the trapping unit information Idt. In a case where the trapping unit information Idt is information indicating that the substance trapping unit 511 is operating, the determination unit 10 may be configured to correct the determination result Rd to a determination result Rd indicating that the infection risk degree Ifr is lower, as compared to a case of information indicating that the substance trapping unit is not operating.

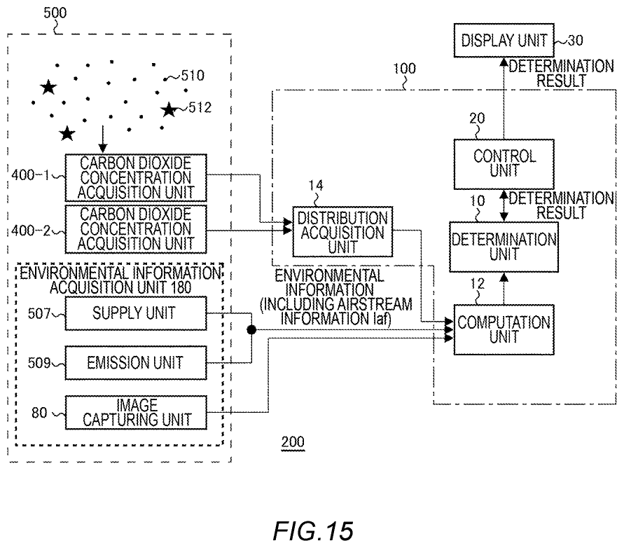

FIG. 15 is a block diagram showing another example of the infection risk determination system 200 according to one embodiment of the present invention. In the infection risk determination system 200 of the present example, the determination apparatus 100 further comprises a distribution acquisition unit 14. In the infection risk determination system 200 of the present example, two carbon dioxide concentration acquisition units 400 are provided in the determination target 500. In these respects, the infection risk determination system 200 of the present example is different from the infection risk determination system 200 shown the FIG. 10. The distribution acquisition unit 14 is configured to acquire a concentration distribution of CO₂ (carbon dioxide) 510 in the determination target 500.

In the determination target 500, the plurality of carbon dioxide concentration acquisition units 400 may be arranged at locations different from each other. In the determination target 500, the plurality of carbon dioxide concentration acquisition units 400 may be arranged at heights different from each other. As shown in FIG. 13, in the present example, the two carbon dioxide concentration acquisition units 400 are provided at heights different from each other.

The distribution acquisition unit 14 may be configured to acquire a concentration distribution of CO₂ (carbon dioxide) 510, based on a plurality of concentrations of CO₂ (carbon dioxide) 510 measured by the plurality of carbon dioxide concentration acquisition units 400. The determination unit 10 may be configured to correct the determination result Rd of the infection risk degree Ifr, based on the concentration distribution of CO₂ (carbon dioxide) 510 acquired by the distribution acquisition unit 14.

The concentration of CO₂ (carbon dioxide) may be different, depending on locations in the internal space 508 (refer to FIG. 11). For this reason, the determination unit 10 corrects the determination result Rd of the infection risk degree Ifr, based on the concentration distribution of CO₂ (carbon dioxide) 510, so that the infection risk degree Ifr is likely to be determined more correctly.

Since CO₂ (carbon dioxide) is heavier than air, CO₂ (carbon dioxide) is likely to stay in the lower of the internal space 508. For this reason, the concentration of CO₂ (carbon dioxide) 510 is likely to be higher in the lower than in the upper of the internal space 508. For this reason, when the distribution acquisition unit 14 acquires a distribution of concentrations of CO₂ (carbon dioxide) 510, based on the plurality of concentrations of CO₂ (carbon dioxide) 510 measured by the plurality of carbon dioxide concentration acquisition units 400 arranged at locations different from each other, the distribution of concentrations of CO₂ (carbon dioxide) 510 is likely to reflect a distribution in a height direction in the internal space 508. For this reason, the determination unit 10 corrects the determination result Rd of the infection risk degree Ifr, based on the distribution of concentrations of CO₂ (carbon dioxide) 510, so that the infection risk degree Ifr is likely to be determined more correctly.

Since the infectious particles IPa are heavier than CO₂ (carbon dioxide) 510, mobility by diffusion of the infectious particles IPa is likely to be smaller than mobility by diffusion by CO₂ (carbon dioxide) 510. For this reason, in the internal space 508, the infectious particles IPa are more susceptible to airstream than CO₂ (carbon dioxide) 510. For this reason, in the internal space 508, a concentration distribution of CO₂ (carbon dioxide) 510 and a concentration distribution of the infectious particles IPa may be different. The determination unit 10 may also be configured to correct the determination result Rd of the infection risk degree Ifr, based on the distribution of concentration of CO₂ (carbon dioxide) 510 and the airstream information Iaf. Thereby, in a case where the concentration distribution of CO₂ (carbon dioxide) 510 and the concentration distribution of the infectious particles IPa are different, the infection risk degree Ifr is likely to be determined more correctly.

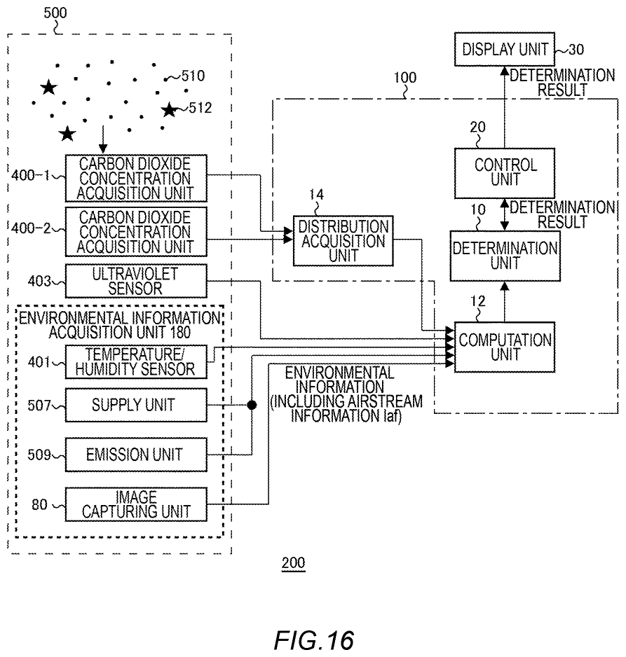

FIG. 16 is a block diagram showing another example of the infection risk determination system 200 according to one embodiment of the present invention. In the infection risk determination system 200 of the present example, a temperature/humidity sensor 401 and an ultraviolet sensor 403 are further provided in the determination target 500. In this respect, the infection risk determination system 200 of the present example is different from the infection risk determination system 200 shown the FIG. 15. As shown in FIG. 13, in the present example, the temperature/humidity sensor 401 is provided on the desk 501, and the ultraviolet sensor 403 is provided on the wall portion 504.

The environmental information Ie may further include at least one of a temperature and a humidity in the internal space 508 (refer to FIG. 12). The temperature is referred to as temperature T. The humidity is referred to as humidity H. The temperature T and the humidity H may be measured by the temperature/humidity sensor 401.

A life-span of the infection source 512 may depend on at least one of the temperature T and the humidity H. In a case where the infection source 512 is a SARS-CoV-2 virus (so-called new corona virus), the life-span of the infection source 512 is more likely to be long as a deviation from a range of a predetermined temperature T is larger, and is more likely to be long as a deviation from a range of a predetermined humidity H is larger. The predetermined humidity H is a range of relative humidity 60%, for example. The relative humidity refers to a ratio of water vapor contained in the air.

The determination unit 10 may be configured to correct the determination result Rd of the infection risk degree Ifr, based on at least one of the temperature T and the humidity H in the internal space 508. This makes it easier to determine the infection risk degree Ifr more correctly.

The determination unit 10 may be configured to correct the determination result Rd of the infection risk degree Ifr, based on an intensity of the ultraviolet measured by the ultraviolet sensor 403. In a case where the infection source 512 is present under environments where the ultraviolet is irradiated, activity of the infection source 512 is likely to depend on the intensity of the ultraviolet. The activity of the infection source 512 refers to a degree that the infection source 512 infects the living body 90. In a case where the infection source 512 is a SARS-CoV-2 virus (so-called new corona virus), the infection source 512 is more likely to be inactivated as the ultraviolet is more intense. For this reason, the determination unit 10 corrects the determination result Rd of the infection risk degree Ifr, based on the intensity of the ultraviolet measured by the ultraviolet sensor 403, so that the infection risk degree Ifr is likely to be determined more correctly.

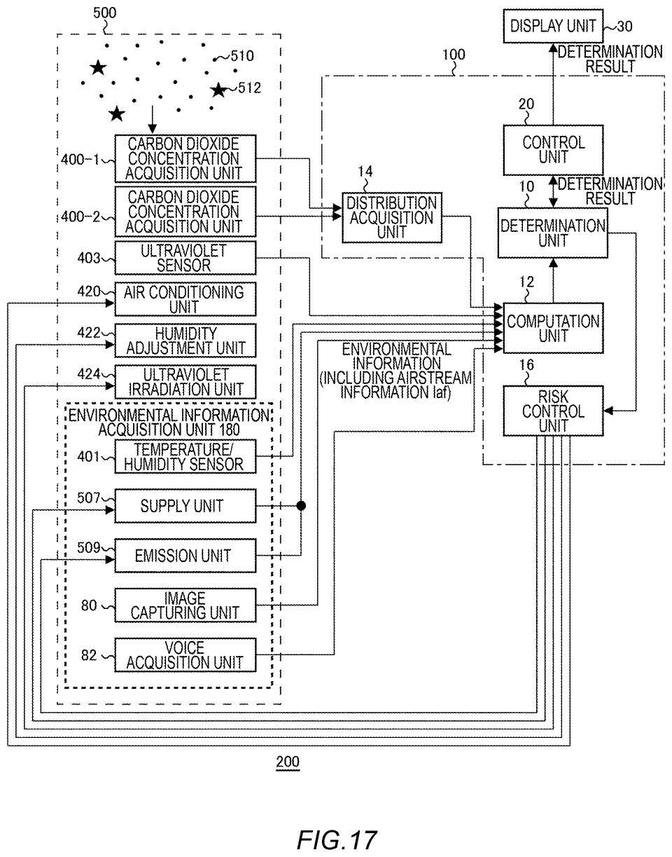

FIG. 17 is a block diagram showing another example of the infection risk determination system 200 according to one embodiment of the present invention. In the infection risk determination system 200 of the present example, an air conditioning unit 420, a humidity adjustment unit 422, an ultraviolet irradiation unit 424 and a voice acquisition unit 82 are further arranged in the determination target 500. The infection risk determination system 200 of the present example further comprises a risk control unit 16. In these respects, the infection risk determination system 200 of the present example is different from the infection risk determination system 200 shown the FIG. 16.

The air conditioning unit 420 is configured to condition a temperature T in the internal space 508. The air conditioning unit 420 is, for example, an air conditioner. Note that, in a case where the air conditioning unit 420 has a blowing function, the supply unit 507 may not be arranged in the determination target 500. The humidity adjustment unit 422 is configured to adjust a humidity H in the internal space 508. The humidity adjustment unit 422 is, for example, a humidifier, and may also be an air conditioner having a humidity adjusting function. The ultraviolet irradiation unit 424 is configured to irradiate the internal space 508 with ultraviolet. The ultraviolet irradiation unit 424 may be an ultraviolet irradiation machine such as an LED type or a lamp type, or may also be sunlight.

The risk control unit 16 is configured to control at least one of the airstream in the internal space 508 (refer to FIG. 11), the temperature T in the internal space 508, the humidity H in the internal space 508, the intensity of ultraviolet in the internal space 508 and the amount of the substance included in the gas in the internal space 508, based on the determination result Rd of the infection risk degree Ifr by the determination unit 10. The risk control unit 16 may be configured to control at least one of the airstream, the temperature T, the humidity H, the intensity of ultraviolet and the amount of the substance included in the gas so that the infection risk degree Ifr decreases, based on the determination result Rd. This not only allows the determination unit 10 to output the determination result Rd of the infection risk degree Ifr, but also allows the infection risk degree Ifr to decrease.

When it is determined by the determination unit 10 that the infection risk degree Ifr in the determination target 500 is high, the risk control unit 16 may be configured to increase at least one of a supply amount of a gas outside the internal space 508 by the supply unit 507 and an emission amount of a gas in the internal space 508 by the emission unit 509. This makes it easier to decrease the infection risk degree Ifr. Even if the determination unit 10 determines that the infection risk degree Ifr at the determination target 500 is high, at least one of the supply of gas outside the internal space 508 by the supply unit 507 and the emission amount of gas in the internal space 508 by the emission unit 509 may not be increased depending on the external conditions. The external condition is, for example, a condition of high pollen counts.

When it is determined by the determination unit 10 that the infection risk degree Ifr in the determination target 500 is high, the risk control unit 16 may be configured to control the air conditioning unit 420 so that the temperature T in the internal space 508 becomes a predetermined temperature. This makes it easier to decrease the infection risk degree Ifr. In a case where the infection source 512 is a SARS-CoV-2 virus (so-called new corona virus), the predetermined temperature is, for example, 20° C. or higher and 25° C. or lower.

When it is determined by the determination unit 10 that the infection risk degree Ifr in the determination target 500 is high, the risk control unit 16 may be configured to control the humidity adjustment unit 422 so that the humidity H in the internal space 508 becomes a predetermined humidity. This makes it easier to decrease the infection risk degree Ifr. In a case where the infection source 512 is a SARS-CoV-2 virus (so-called new corona virus), the predetermined humidity is, for example, 40% or more.

When it is determined by the determination unit 10 that the infection risk degree Ifr in the determination target 500 is high, the risk control unit 16 may be configured to turn on the ultraviolet irradiation unit 424. This makes it easier to decrease the infection risk degree Ifr. The risk control unit 16 may also be configured to control the intensity of ultraviolet that is irradiated by the ultraviolet irradiation unit 424. In a case where the ultraviolet irradiation unit 424 is sunlight, the risk control unit 16 may also be configured to control an amount of sunlight that is irradiated to the internal space 508 by controlling an opening/closing degree of the ceiling portion 506 in the internal space 508. Thereby, in a case where the infection source 512 is a SARS-CoV-2 virus (so-called new corona virus), the infection source 512 is likely to be inactivated. This makes it easier to decrease the infection risk degree Ifr. The risk control unit 16 may also be configured to operate the ultraviolet irradiation unit 424, when it is detected that the living body 90 is absent.

When it is determined by the determination unit 10 that the infection risk degree Ifr in the determination target 500 is high, the risk control unit 16 may be configured to control the substance trapping unit 511 to decrease the infection risk degree Ifr. The risk control unit 16 may be configured to control the substance trapping unit 511 so that the substance trapping unit 511 sucks the gas accommodated in the internal space 508. The substance trapping unit 511 may be configured to trap a substance (mote, dust, virus and the like) contained in the gas accommodated in the internal space 508. This makes it easier to decrease the infection risk degree Ifr.

The risk control unit 16 may also be included in the control unit 20. The determination apparatus 100 may not comprise the risk control unit 16, and the control unit 20 may also be configured to control at least one of the airstream in the internal space 508 (refer to FIG. 11), the temperature T in the internal space 508, the humidity H in the internal space 508, and the intensity of ultraviolet in the internal space 508.

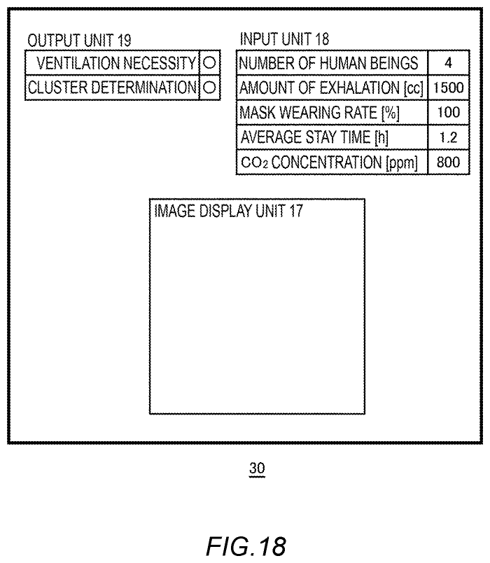

FIG. 18 shows an example of a display aspect on the display unit 30. The display unit 30 of the present example includes an input unit 18, an output unit 19 and an image display unit 17. The user of the infection risk determination system 200 may input the environmental information Ie from the input unit 18. The determination result Rd of the infection risk degree Ifr may be output to the output unit 19 or may be displayed on the image display unit 17. In the present example, the determination result Rd is displayed on the output unit 19 with information having readability (for example, characters, numbers, and the like), and is displayed on the image display unit 17 with information having visual recognition (for example, a graph, a figure, and the like). Note that, the input unit 18 and the output unit 19 may also be included in the image display unit 17.

The environmental information Ie based on an image captured by the image capturing unit 80 may also be automatically input to the input unit 18. For example, for an item of ‘average stay time’, a staying time based on the staying time information Is may be automatically input, and for an item of ‘CO₂ concentration’, a concentration of CO₂ (carbon dioxide) 510 measured by the carbon dioxide concentration acquisition unit 400 may also be automatically displayed.

The display unit 30 may be configured to display, as a control state by the risk control unit 16, a control state of at least one of the airstream in the internal space 508, the temperature T in the internal space 508, the humidity H in the internal space 508, the intensity of ultraviolet in the internal space 508 and the amount of the substance included in the gas in the internal space 508. This makes the operational state of the risk control unit 16 visible. Visualization of the operational state of the risk control unit 16 enables control of the number of occupants in accordance with the actual operational state of the determination target 500. This makes it possible to operate the determination target 500 economically. As a result, the asset value of the determination target 500 can be improved. An effect as a result of the ultraviolet irradiation unit 424 being operated may also be displayed on the display unit 30. The effect as a result of the ultraviolet irradiation unit 424 being operated may be a change of the infection risk degree Ifr before and after the ultraviolet irradiation unit 424 is operated.

An image captured by the image capturing unit 80 may also be displayed on the image display unit 17. The image and the determination result Rd of the infection risk degree Ifr may also be displayed together on the image display unit 17. The image shown in FIG. 1 captured by the image capturing unit 80 may also be displayed on the image display unit 17.

The display unit 30 may be configured to display the determination result Rd of the infection risk degree Ifr for a predetermined time since the image capturing unit 80 acquires an image of the determination target 500, in which the number information In of the living body 90 is zero. The image of the determination target 500, in which the number information In of the living body 90 is zero, refers to an image in which the living body 90 has not been image-captured. The predetermined time since an image in which the number information In is zero is acquired may be a time since an image in which the number information In is not zero is changed into the image in which the number information In is zero. The predetermined time since an image is acquired is a time for which the infection risk degree Ifr by the remaining infection source 512 remains.

In the present example, since the determination result Rd of the infection risk degree Ifr is displayed on the display unit 30 for the predetermined time since an image in which the number information In is zero is acquired, the infection risk determination system 200 can notify the living body 90 who intends to newly enter the internal space 508 of the infection risk degree Ifr in the determination target 500. The display unit 30 may stop displaying the determination result Rd after a predetermined time has elapsed since the image with the number information In is zero is acquired. If a new living body 90 enters the internal space 508, an image can be acquired where the number information In is greater than 1. If an image is acquired where the number information In is greater than or equal to 1, the display unit 30 may again display the determination result Rd.

The display unit 30 may be configured to display environmental information Ie, which has the greatest degree of contribution to the determination result Rd of the infection risk degree Ifr, among the environmental information Ie. The display unit 30 may be configured to display environmental information Ie, which has the greatest degree of contribution to the determination result Rd of the infection risk degree Ifr, among the sound information Iv, the number information In, the body temperature information It, the exposure information Ip, the distance information Id, the location information IL, the staying time information Is and the motion information Im. The display unit 30 may also be configured to display the environmental information Ie, which has the greatest degree of contribution to the determination result Rd of the infection risk degree Ifr, among the environmental information Ie, and to display the environmental information Ie having the greatest degree of contribution in an aspect different from the other environmental information. The different aspect is such that a character is made bold, a color of a character is changed, a character is blinked and the like, for example.

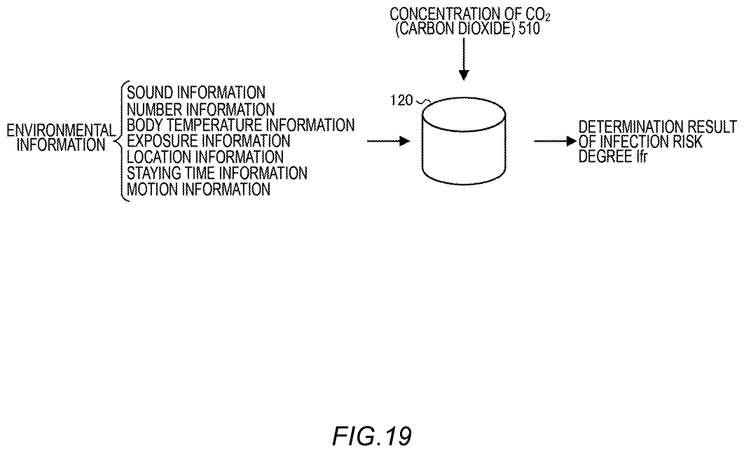

FIG. 19 shows an example of a derivation method of the determination result Rd. When the environmental information Ie and the concentration of CO₂ (carbon dioxide) 510 are input, a determination inference model 120 is configured to output the determination result Rd of the infection risk degree Ifr with respect to the environmental information Ie and the concentration of CO₂ (carbon dioxide) 510. The environmental information Ie includes at least one of the sound information Iv, the number information In, the body temperature information It, the exposure information Ip, the distance information Id, the location information IL, the staying time information Is, and the motion information Im. The determination apparatus 100 may comprise the determination inference model unit 120. The determination inference model 120 may be generated by machine-learning the environmental information Ie and the concentration of CO₂ (carbon dioxide) 510.

It is assumed that at least one of one environmental information Ie and one concentration of CO₂ (carbon dioxide) 510 is currently input to the determination inference model 120. In the case where at least one of one environmental information Ie and one concentration of CO₂ (carbon dioxide) 510 is currently input to the determination inference model 120, when at least one of the other environmental information Ie and the other concentrations of CO₂ (carbon dioxide) 510 is input, the display unit 30 may display a change of the determination result Rd of the infection risk degree Ifr. Thereby, the user of the infection risk determination system 200 can recognize the change of the determination result Rd when at least one of at least one of the environmental information Ie and the concentration of CO₂ (carbon dioxide) 510 has been changed from the current environmental information Ie and concentration of CO₂ (carbon dioxide) 510.

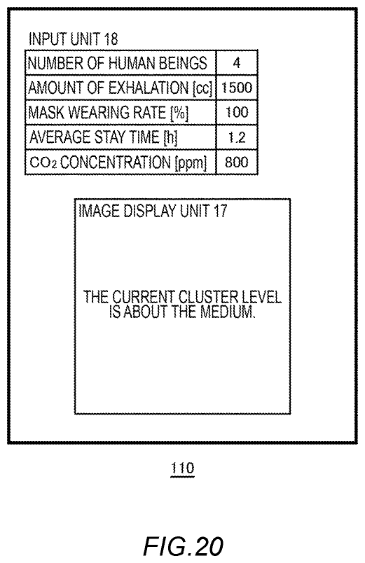

FIG. 20 shows an example of a display aspect on the mobile terminal 110 (refer to FIG. 4). In FIG. 20, the input unit 18 provided to the mobile terminal 110 is shown. The user of the infection risk determination system 200 may also input the environmental information Ie from the input unit 18 provided to the mobile terminal 110. The mobile terminal 110 may be provided with the image display unit 17. The determination result Rd of the infection risk degree Ifr may be displayed on the image display unit 17. In the present example, the determination result Rd “the current cluster level is about the medium.” is displayed on the image display unit 17. An effect as a result of the ultraviolet irradiation unit 424 being operated may also be displayed on the image display unit 17.

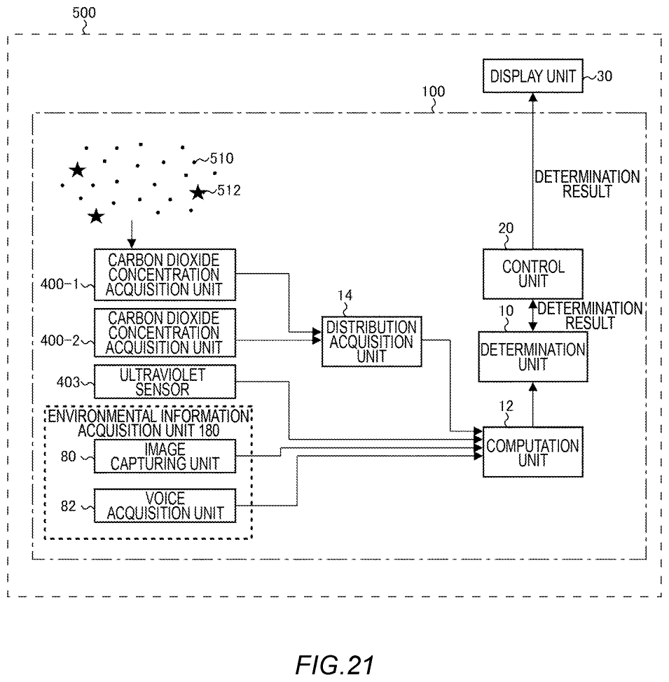

FIG. 21 shows an example of the determination apparatus 100 according to one embodiment of the present invention. In the present example, the determination apparatus 100 comprises the carbon dioxide concentration acquisition unit 400, the image capturing unit 80, the voice acquisition unit 82, and the ultraviolet sensor 403. The determination apparatus 100 (refer to FIG. 1) arranged in the determination target 500 may also comprise the carbon dioxide concentration acquisition unit 400, the image capturing unit 80, the voice acquisition unit 82, and the ultraviolet sensor 403. The determination apparatus 100 may also further comprise the temperature/humidity sensor 401 (refer to FIG. 17). The determination apparatus 100 may also further comprise the display unit 30, and may display an effect as a result of the substance trapping unit 511 being operated.

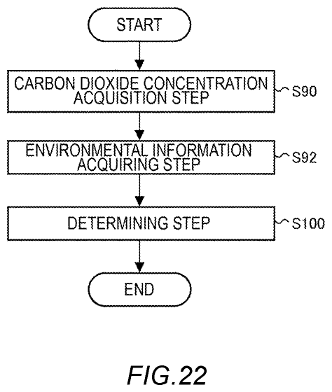

FIG. 22 is a flowchart showing an example of a determination method according to one embodiment of the present invention. The determination method according to one embodiment of the present invention is described by taking the infection risk determination system 200 shown in FIG. 3 as an example A carbon dioxide concentration acquisition step S90 is a step of, by one or a plurality of carbon dioxide concentration acquisition units 400, measuring a concentration of CO₂ (carbon dioxide) 510 in the determination target 500. An environmental information acquiring step S92 is a step of, by the environmental information acquisition unit 180, acquiring the environmental information Ie in the determination target 500. A determining step S100 is a step of, by the determination unit 10, determining the infection risk degree Ifr that one or a plurality of living bodies 90 present in the determination target 500 will be infected with the infection source 512 present in the determination target 500, based on the concentration of CO₂ (carbon dioxide) 510 and the environmental information Ie.

In the determination method shown in FIG. 22, in the determining step S100, the infection risk degree Ifr that the living body 90 will be infected with the infection source 512 is determined. For this reason, the user of the infection risk determination system 200 can recognize the infection risk degree Ifr.

The environmental information Ie may be at least one of the sound information Iv, the number information In, the body temperature information It, the exposure information Ip, the distance information Id, the location information IL, the staying time information Is, and the motion information Im. The exposure information Ip may include the information about a type of a mask configured to cover a nose or mouth of the living body 90.

The environmental information Ie may be at least one of information based on an image of the determination target 500 captured by the image capturing unit 80 (refer to FIG. 2) and information based on a sound of the living body 90 acquired by the voice acquisition unit 82 (refer to FIG. 2). The number information In, the body temperature information It, the exposure information Ip, the distance information Id, the location information IL, the staying time information Is and the motion information Im may be information based on an image captured by the image capturing unit 80. The sound information Iv may be information based on a sound of the living body 90 acquired by the voice acquisition unit 82.

The determining step S100 may be a step of, by the determination unit 10, further determining the living body 90, which is an information source of the sound information Iv, among the plurality of living bodies 90, based on an image of the determination target 500 captured by the image capturing unit 80 (refer to FIG. 5) and a sound of the living body 90 acquired by the voice acquisition unit 82 (refer to FIG. 5). The living body 90, which is an information source of the sound information Iv, among the plurality of living bodies 90 is preferably determined based on a plurality of images captured by the plurality of image capturing units 80 and a plurality of voices acquired by the plurality of voice acquisition units.

The environmental information Ie may include the infection information Ifi as to whether the living body 90 is infected with the infection source 512. The determining step S100 may be a step of, by the determination unit 10, determining the infection risk degree Ifr, based on at least one of a sound-producing time and a sound-producing amount of the living body 90, which is an information source of the sound information Iv, when the environmental information Ie includes the infection information Ifi.

The living body 90 may be a human being. The environmental information Ie may further include at least one of a sex, an age, a height, a chronic disease history, and an illness history to an infectious disease of the human being. The environmental information Ie may further include the statistical information Ist regarding an infection status of the living body 90 infected with the infection source 512.

The environmental information Ie may further include the airstream information Iaf in the internal space 508 (refer to FIG. 9). The environmental information Ie may further include the information about the CO₂ (carbon dioxide) emission unit 505 (refer to FIG. 11) configured to emit CO₂ (carbon dioxide) 510 exceeding the predetermined amount in the internal space 508. The environmental information Ie may further include at least one of the temperature T and the humidity H in the internal space 508 (refer to FIG. 9).

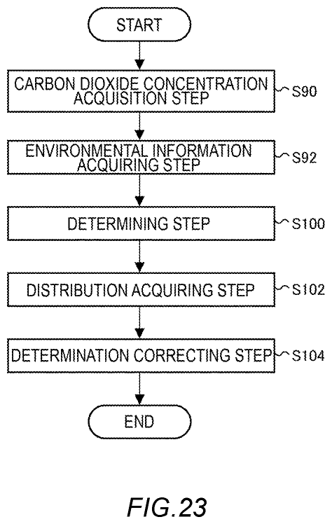

FIG. 23 is a flowchart showing another example of the determination method according to one embodiment of the present invention. The determination method of the present example is different from the determination method shown in FIG. 22, in that the determination method further comprises a distribution acquiring step S102 and a determination correcting step S104. The determination method shown in FIG. 23 is described by taking the infection risk determination system 200 shown in FIG. 15 as an example.

In the determination target 500, the plurality of carbon dioxide concentration acquisition units 400 may be arranged at locations different from each other. The plurality of carbon dioxide concentration acquisition units 400 are preferably arranged at heights different from each other. The distribution acquiring step S102 is a step of, by the distribution acquisition unit 14, acquiring a distribution of concentrations of CO₂ (carbon dioxide) 510 in the determination target 500, based on a plurality of concentrations of CO₂ (carbon dioxide) 510 measured by the plurality of carbon dioxide concentration acquisition units 400. The determination correcting step S104 is a step of, by the determination unit 10, correcting the determination result Rd of the infection risk degree Ifr, based on the distribution of concentrations of CO₂ (carbon dioxide) 510.

The concentration of CO2 (carbon dioxide) may be different, depending on locations in the internal space 508 (refer to FIG. 11). For this reason, the determination result Rd of the infection risk degree Ifr is corrected, based on the distribution of concentrations of CO2 (carbon dioxide) 510, so that the infection risk degree Ifr is likely to be determined more correctly.

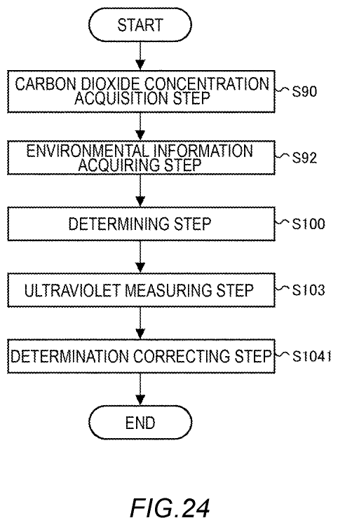

FIG. 24 is a flowchart showing another example of the determination method according to one embodiment of the present invention. The determination method of the present example is different from the determination method shown in FIG. 22, in that the determination method further comprises an ultraviolet acquiring step S103 and a determination correcting step S1041. The determination method shown in FIG. 24 is described by taking the infection risk determination system 200 shown in FIG. 16 as an example.

The ultraviolet measuring step S103 is a step of, by the ultraviolet sensor 403, measuring ultraviolet in the determination target 500. The determination correcting step S1041 is a step of, by the determination unit 10, correcting the determination result Rd of the infection risk degree Ifr, based on the intensity of the ultraviolet measured by the ultraviolet sensor 403.

In a case where the infection source 512 is present under environments where the ultraviolet is irradiated, the activity of the infection source 512 is likely to depend on the intensity of the ultraviolet. The activity of the infection source 512 refers to a degree that the infection source 512 infects the living body 90. For this reason, the determination result Rd of the infection risk degree Ifr is corrected, based on the intensity of the ultraviolet measured by the ultraviolet sensor 403, so that the infection risk degree Ifr is likely to be determined more correctly.

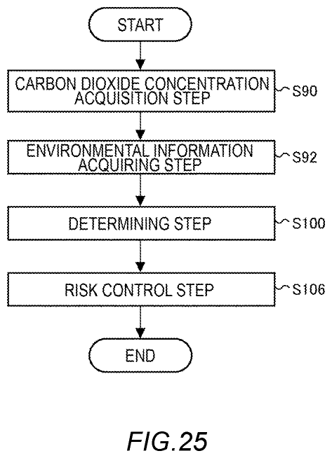

FIG. 25 is a flowchart showing another example of the determination method according to one embodiment of the present invention. The determination method of the present example is different from the determination method shown in FIG. 22, in that the determination method further comprises a risk control step S106. The determination method shown in FIG. 25 is described by taking the infection risk determination system 200 shown in FIG. 17 as an example.

The risk control step S106 is a step of, by the risk control unit 16, controlling at least one of the airstream in the internal space 508 (refer to FIG. 9), the temperature T in the internal space 508, the humidity H in the internal space 508, the intensity of ultraviolet in the internal space 508 and the amount of the substance included in the gas in the internal space 508, based on the determination result Rd of the infection risk degree Ifr by the determination unit 10. The risk control step S106 may be a step of, by the risk control unit 16, controlling, based on the determination result Rd of the infection risk degree Ifr by the determination unit 10, at least one of the airstream, the temperature T, the humidity H, the intensity of ultraviolet in the internal space 508 and the amount of the substance included in the gas in the internal space 508 so that the infection risk degree Ifr decreases. Thereby, the infection risk degree Ifr that the living body 90 will be infected with the infection source 512 is likely to decrease.

A variety of embodiments of the present invention may be described with reference to flowcharts and block diagrams. In the various embodiments of the present invention, blocks may represent (1) steps of processes in which operations are performed or (2) sections of apparatuses responsible for performing operations.

Certain steps may be executed by dedicated circuitry, programmable circuitry or processors. Certain sections may be implemented by dedicated circuitry, programmable circuitry or processors. The programmable circuitry and the processors may be supplied together with computer-readable instructions. The computer-readable instructions may be stored on computer-readable media.

The dedicated circuitry may include at least one of a digital hardware circuit and an analog hardware circuit. The dedicated circuitry may include at least one of an integrated circuit (IC) and a discrete circuit. The programmable circuitry may include a hardware circuit of logical AND, logical OR, logical XOR, logical NAND, logical NOR or other logical operations. The programmable circuitry may include a reconfigurable hardware circuit including a memory element such a flip-flop, a register, a field programmable gate array (FPGA) and a programmable logic array (PLA), and the like.

Computer-readable media may include any tangible device that can store instructions to be executed by a suitable device. Computer-readable media include the tangible device, so that computer-readable media having instructions to be stored in the device comprise an article of manufacture including instructions that can be executed to provide means for performing operations specified in the flowcharts or block diagrams.

Examples of computer-readable media may include an electronic storage medium, a magnetic storage medium, an optical storage medium, an electromagnetic storage medium, a semiconductor storage medium, and the like. More specific examples of computer-readable media may include a floppy (registered trademark) disk, a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disk (DVD), a BLU-RAY (registered trademark) disk, a memory stick, an integrated circuit card, and the like.

Computer-readable instructions may include any one of assembler instructions, instruction-set-architecture (ISA) instructions, machine instructions, machine dependent instructions, microcode, firmware instructions, state-setting data, source code and object code. The source code and the object code may be described in any combination of one or a plurality of programming languages, including an object oriented programming language and a conventional procedural programming language. The object oriented programming language may be, for example, Smalltalk (registered trademark), JAVA (registered trademark), C++, or the like. The procedural programming language may be, for example, a ‘C’ programming language.

Computer-readable instructions may be provided to a processor of a general purpose computer, a special purpose computer, or other programmable data processing apparatus, or to a programmable circuitry, locally or via a local area network (LAN), wide area network (WAN) such as the Internet, and the like. A processor of a general purpose computer, a special purpose computer, or other programmable data processing apparatus or a programmable circuitry may be configured to execute the computer-readable instructions so as to provide means for executing operations specified in the flowcharts shown in FIGS. 22 to 25 or the block diagrams shown in FIGS. 3 to 8, FIG. 10, FIG. 12, FIGS. 14 to 17 and FIG. 21. Examples of a processor include a computer processor, a processing unit, a microprocessor, a digital signal processor, a controller, a microcontroller, and the like.

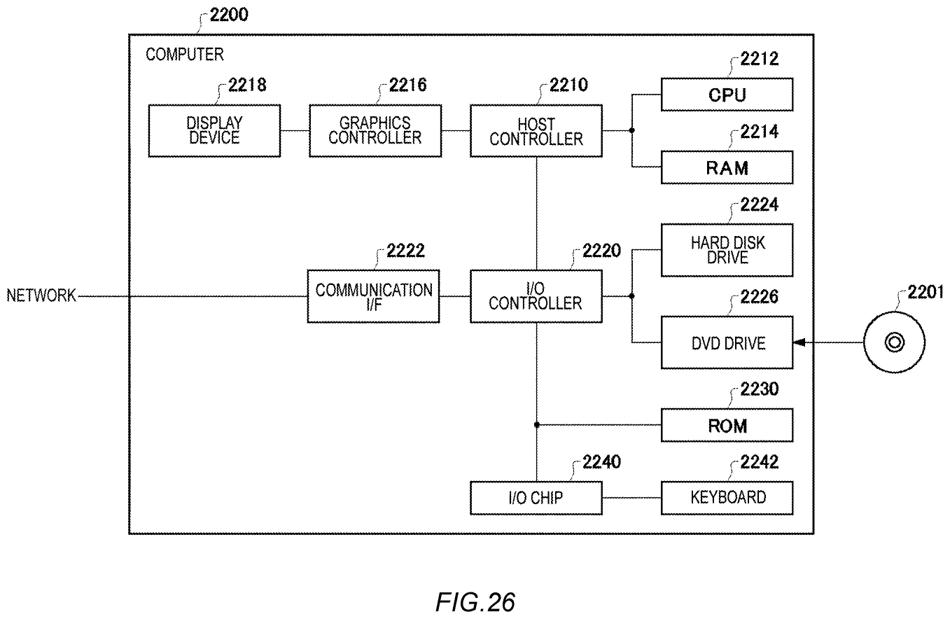

FIG. 26 shows an example of a computer 2200 in which the determination apparatus 100 or the infection risk determination system 200 according to one embodiment of the present invention may be entirely or partially embodied. A program that is installed in the computer 2200 can cause the computer 2200 to function as or execute operations associated with the determination apparatus 100 of the embodiment of the present invention or one or a plurality of sections of the determination apparatus 100 or the infection risk determination system 200, or cause the computer 2200 to execute the respective steps (refer to FIGS. 22 to 25) of the determination method of the present. The program may be executed by a CPU 2212 so as to cause the computer 2200 to execute certain operations associated with some or all of the flowcharts (FIGS. 22 to 25) and blocks of block diagrams (FIGS. 3 to 8, FIG. 10, FIG. 12, FIGS. 14 to 17 and FIG. 21) described herein.

The computer 2200 according to one embodiment of the present invention includes the CPU 2212, a RAM 2214, a graphic controller 2216 and a display device 2218. The CPU 2212, the RAM 2214, the graphic controller 2216 and the display device 2218 are mutually connected by a host controller 2210. The computer 2200 further includes input and output units such as a communication interface 2222, a hard disk drive 2224, a DVD-ROM drive 2226 and an IC card drive. The communication interface 2222, the hard disk drive 2224, the DVD-ROM drive 2226, the IC card drive and the like are connected to the host controller 2210 via an input and output controller 2220. The computer further includes legacy input and output units such as a ROM 2230 and a keyboard 2242. The ROM 2230, the keyboard 2242, and the like are connected to the input and output controller 2220 via an input and output chip 2240.

The CPU 2212 is configured to operate according to programs stored in the ROM 2230 and the RAM 2214, thereby controlling each unit. The graphic controller 2216 is configured to acquire image data generated by the CPU 2212 on a frame buffer or the like provided in the RAM 2214 or in the RAM 2214, and to cause the image data to be displayed on the display device 2218.

The communication interface 2222 is configured to communicate with other electronic devices via a network. The hard disk drive 2224 is configured to store programs and data that are used by the CPU 2212 within the computer 2200. The DVD-ROM drive 2226 is configured to read programs or data from a DVD-ROM 2201, and to provide the hard disk drive 2224 with the read programs or data via the RAM 2214. The IC card drive is configured to read programs and data from an IC card, or to write programs and data into the IC card.

The ROM 2230 is configured to store a boot program or the like that is executed by the computer 2200 at the time of activation, or a program depending on hardware of the computer 2200. The input and output chip 2240 may also be configured to connect various input and output units to the input and output controller 2220 via a parallel port, a serial port, a keyboard port, a mouse port and the like.

A program is provided by a computer-readable medium such as the DVD-ROM 2201 or the IC card. The program is read from the computer-readable medium, is installed into the hard disk drive 2224, the RAM 2214 or the ROM 2230, which are also examples of the computer-readable medium, and is executed by the CPU 2212. Information processing described in these programs is read into the computer 2200, resulting in cooperation between the programs and the various types of hardware resources described above. An apparatus or method may be constituted by realizing the operation or processing of information according to a use of the computer 2200.

For example, when communication is performed between the computer 2200 and an external device, the CPU 2212 may be configured to execute a communication program loaded onto the RAM 2214 to instruct the communication interface 2222 for communication processing, based on processing described in the communication program. The communication interface 2222 is configured, under control of the CPU 2212, to read transmission data stored on a transmission buffer processing area provided in a recording medium such as the RAM 2214, the hard disk drive 2224, the DVD-ROM 2201 or the IC card, and to transmit the read transmission data to a network or to write reception data received from the network to a reception buffer processing area or the like provided on the recording medium.

The CPU 2212 may be configured to cause all or a necessary portion of a file or a database, which has been stored in an external recording medium such as the hard disk drive 2224, the DVD-ROM drive 2226 (DVD-ROM 2201) and the IC card, to be read into the RAM 2214. The CPU 2212 may be configured to execute various types of processing on the data on the RAM 2214. Next, the CPU 2212 may be configured to write the processed data back to the external recording medium.

Various types of information, such as various types of programs, data, tables, and databases, may be stored in the recording medium and may be subjected to information processing. The CPU 2212 may be configured to execute, on the data read from the RAM 2214, various types of processing including various types of operations, processing of information, conditional judgment, conditional branching, unconditional branching, search or replacement of information and the like described in the present disclosure and specified by instruction sequences of the programs. The CPU 2212 may be configured to write results back to the RAM 2214.

The CPU 2212 may also be configured to search for information in a file, a database, and the like in the recording medium. For example, when a plurality of entries, each having an attribute value of a first attribute associated with an attribute value of a second attribute, is stored in the recording medium, the CPU 2212 may be configured to search for an entry having a designated attribute value of the first attribute that matches a condition from the plurality of entries, and to read the attribute value of the second attribute stored in the entry, thereby acquiring the attribute value of the second attribute associated with the first attribute that satisfies a predetermined condition.

The programs or software modules described above may be stored on the computer 2200 or in a computer-readable medium of the computer 2200. A recording medium such as a hard disk or a RAM provided in a server system connected to a dedicated communication network or the Internet can be used as a computer-readable medium. The program may be provided to the computer 2200 by the recording medium.

While the embodiments of the present invention have been described, the technical scope of the invention is not limited to the above described embodiments. It is apparent to persons skilled in the art that various alterations and improvements can be added to the above-described embodiments. It is also apparent from the scope of the claims that the embodiments added with such alterations or improvements can be included in the technical scope of the invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

### EXPLANATION OF REFERENCES

10: determination unit, 12: computation unit, 14: distribution acquisition unit, 16: risk control unit, 17: image display unit, 18: input unit, 19: output unit, 20: control unit, 30: display unit, 80: image capturing unit, 82: voice acquisition unit, 90: living body, 91: mask, 100: determination apparatus, 110: mobile terminal, 120: determination inference model, 180: environmental information acquisition unit, 200: infection risk determination system, 400: carbon dioxide concentration acquisition unit, 401: temperature/humidity sensor, 403: ultraviolet sensor, 420: air conditioning unit, 422: humidity adjustment unit, 424: ultraviolet irradiation unit, 500: determination target, 501: desk, 502: floor portion, 504: wall portion, 505: emission unit, 506: ceiling portion, 507: supply unit, 508: internal space, 509: emission unit, 510: CO₂ (carbon dioxide), 511: substance trapping unit, 512: infection source, 2200: computer, 2201: DVD-ROM, 2210: host controller, 2212: CPU, 2214: RAM, 2216: graphic controller, 2218: display device, 2220: input and output controller, 2222: communication interface, 2224: hard disk drive, 2226: DVD-ROM drive, 2230: ROM, 2240: input and output chip, 2242: keyboard

What is claimed is:

1. An infection risk determination system comprising:
a determination apparatus, including a determination unit configured to determine an infection risk degree that one living body or a plurality of living bodies present in a determination target will be infected with an infection source present in the determination target, based on a carbon dioxide concentration in the determination target with an internal space for accommodating a gas containing carbon dioxide and environmental information in the determination target;
a risk control unit, configured to control at least one of an airstream in the internal space, a temperature of the internal space, a humidity of the internal space, an intensity of ultraviolet radiation in the internal space, and an amount of substances in a gas in the internal space based on a determination result of the infection risk degree by the determination unit; and
a display unit, configured to display a control state by the risk control unit,
wherein the environmental information includes at least one of sound information of the living body, number information of the living body, body temperature information of the living body, nose or mouth exposure information of the living body, distance information between the plurality of the living bodies, location information of the living bodies, staying time information of the living body, and motion information of the living body.

2. The infection risk determination system according to claim 1, further comprising
an image capturing unit configured to capture an image of the determination target,
wherein the image capturing unit is arranged in the determination target, and
wherein the number information about the living body, the body temperature information about the living body, the exposure information about a nose or mouth of the living body, the distance information between the plurality of living bodies, the location information about the living body, the stay time information about the living body, and the motion information about the living body are information based on the image of the determination target captured by the image capturing unit.

3. The infection risk determination system according to claim 2, further comprising
a voice acquisition unit configured to acquire a sound of the living body.
Wherein the voice acquisition unit is arranged in the determination target,
the sound information about the living body is information based on the sound of the living body acquired by the voice acquisition unit, and
the determination unit is configured to further determine a living body, which is an information source of the sound information, among the plurality of living bodies, based on the image of the determination target captured by the image capturing unit and the sound of the living body acquired by the voice acquisition unit.

4. The infection risk determination system according to claim 1, wherein
the exposure information about a nose or mouth of the living body includes information about a type of a mask configured to cover the nose or mouth of the living body, and
the determination unit is configured to correct a determination result of the infection risk degree, based on the information about the type of the mask.

5. The infection risk determination system according to claim 1, wherein
the living body is a human being,
the environmental information further includes at least one of a sex, an age, a height, a chronic disease history, and an illness history to an infectious disease of the human being, and
the determination unit is configured to correct a determination result of the infection risk degree, based on at least one of the sex, the age, the height, the chronic disease history, and the illness history to an infectious disease of the human being.

6. The infection risk determination system according to claim 1, wherein
the environmental information further includes statistical information regarding an infection status of the living body infected with the infection source, and
the determination unit is configured to correct a determination result of the infection risk degree, based on the statistical information.

7. The infection risk determination system according to claim 1, wherein
the determination unit is configured to correct a determination result of the infection risk degree, based on at least one of the airstream information in the internal space and location information about the living body in the internal space.

8. The infection risk determination system according to claim 1, wherein
the environmental information further includes at least one of information about a carbon dioxide emission unit configured to emit carbon dioxide exceeding a predetermined amount in the internal space and information about a substance trapping unit configured to trap a substance included in the gas, and
the determination unit is configured to correct a determination result of the infection risk degree, based on at least one of the information about the carbon dioxide emission unit and information of the substance trapping unit.

9. The infection risk determination system according to claim 1, further comprising
a carbon dioxide concentration acquisition unit, configured to acquire the carbon dioxide concentration in the internal space,
wherein the carbon dioxide concentration acquisition unit is arranged in the determination target, and
the determination unit is configured to correct a determination result of the infection risk degree, based on a temporal change of the carbon dioxide concentration measured by the carbon dioxide concentration acquisition unit.

10. The infection risk determination system according to claim 9, further comprising a distribution acquisition unit configured to acquire a distribution of carbon dioxide concentration in the determination target, wherein
a plurality of the carbon dioxide concentration acquisition units are arranged at locations different from each other in the determination target,
the distribution acquisition unit is configured to acquire the distribution of carbon dioxide concentrations, based on a plurality of carbon dioxide concentrations measured by a plurality of the carbon dioxide concentration acquisition units, and
the determination unit is configured to correct a determination result of the infection risk degree, based on the distribution of carbon dioxide concentrations.

11. The infection risk determination system according to claim 1, wherein:
the environmental information further includes at least one of a temperature and a humidity in the internal space; and
the determination unit is configured to correct a determination result of the infection risk degree, based on at least one of the temperature and the humidity in the internal space.

12. The infection risk determination system according to claim 1, wherein
the display unit is further configured to display a determination result of the infection risk degree by the determination unit.

13. The infection risk determination system according to claim 1, wherein
the display unit is configured to display the determination result of the infection risk degree for a predetermined time since acquiring an image of the determination target, in which the number information about the living body is zero.

14. The infection risk determination system according to claim 1, wherein
the display unit is configured to display the environmental information, which has the greatest degree of contribution to the determination result of the infection risk degree, among the environmental information.

15. The infection risk determination system according to claim 14, further comprising a determination inference model configured to output the determination result of the infection risk degree with respect to the environmental information and the carbon dioxide concentration when the environmental information and the carbon dioxide concentration are input, wherein
in a case where at least one of the environmental information and the carbon dioxide concentration is input to the determination inference model, when at least one of other environmental information and another carbon dioxide concentrations is input, the display unit is configured to display a change of the determination result of the infection risk degree.

16. An infection risk determination method comprising:
acquiring, by a carbon dioxide concentration acquisition unit, a carbon dioxide concentration in a determination target with an internal space for accommodating a gas containing carbon dioxide;
acquiring, by an environmental information acquisition unit, environmental information in the determination target;
determining, by a determination unit, an infection risk degree that one living body or a plurality of living bodies present in the determination target will be infected with an infection source present in the determination target, based on the carbon dioxide concentration and the environmental information; and
controlling, by a risk control unit, at least one of an airstream in the internal space, a temperature in the internal space, a humidity in the internal space, an intensity of ultraviolet radiation in the internal space, and an amount of substances contained in a gas in the internal space based on a determination result of the infection risk degree by the determination unit; and
displaying, by a display unit, a control state by the risk control unit,
wherein the environmental information includes at least one of sound information of the living body, number information of the living body, body temperature information of the living body, nose or mouth exposure information of the living body, distance information between the plurality of living bodies, location information of the living body, staying time information of the living body, and motion information of the living body.

17. The infection risk determination method according to claim 16, wherein
the environmental information is at least one of information based on an image of the determination target captured by an image capturing unit and information based on a sound of the living body acquired by a voice acquisition unit.

18. The infection risk determination method according to claim 17, wherein
the environmental information includes sound information of the living body, and
the determining is further determining, by the determination unit, a living body, which is an information source of the sound information, among the plurality of living bodies, based on the image of the determination target captured by the image capturing unit and the sound of the living body acquired by the voice acquisition unit.

19. The infection risk determination method according to claim 16, wherein
a plurality of the carbon dioxide concentration acquisition units are arranged at locations different from each other in the determination target,
further comprising:
acquiring, by a distribution acquisition unit, a distribution of carbon dioxide concentrations in the determination target, based on a plurality of carbon dioxide concentrations measured by a plurality of the carbon dioxide concentration acquisition units, and
correcting, by the determination unit, a determination result of the infection risk degree, based on the distribution of carbon dioxide concentrations.

20. A computer-readable medium having recorded thereon a program that, when executed by a computer, causes the computer to execute:
acquiring a carbon dioxide concentration in a determination target with an internal space for accommodating a gas containing carbon dioxide;
acquiring environmental information in the determination target, wherein the environment information includes at least one of sound information of a living body, number information of the living body, body temperature information of the living body, nose or mouth exposure information of the living body, distance information between a plurality of the living bodies, location information of the living bodies, staying time information of the living body, and motion information of the living body;
determining an infection risk degree that one living body or a plurality of living bodies present in the determination target will be infected with an infection source present in the determination target, based on the carbon dioxide concentration and the environmental information;
controlling at least one of an airstream in the internal space, a temperature in the internal space, a humidity in the internal space, an intensity of ultraviolet radiation in the internal space, and an amount of substances contained in a gas in the internal space based on a determination result of the infection risk degree by the determination unit; and
displaying a control state by the risk control unit.
